(19) United States
(12) Patent Application Publication
(10) Pub. No.: US 2024/0324975 A1
(43) Pub. Date: Oct. 3, 2024

(54) PROTECTIVE DEVICE/INSTRUMENT FOR REDUCING RADIATION EXPOSURE AND BURDEN OF PROTECTION

(71) Applicant: Ryutaro WADA, Tokyo (JP)

(72) Inventor: Ryutaro WADA, Tokyo (JP)

(21) Appl. No.: 18/738,062

(22) Filed: Jun. 10, 2024

Related U.S. Application Data
(63) Continuation of application No. PCT/JP2023/003711, filed on Feb. 6, 2023.

(30) Foreign Application Priority Data
Feb. 8, 2022 (JP) 2022-018334
May 1, 2022 (JP) 2022-075633
Aug. 1, 2022 (JP) 2022-123002
Oct. 6, 2022 (JP) 2022-161788
Dec. 22, 2022 (JP) 2022-205553

(51) Int. Cl. A61B 6/10; A61B 6/00; A61B 6/04; A61B 6/42

(52) U.S. Cl. CPC A61B 6/107; A61B 6/0407; A61B 6/42; A61B 6/4405; A61B 6/4452

(57) ABSTRACT

By combining a plurality of protective devices (PD), the present invention makes it possible to allow good transmission of primary X-rays and to reduce the strength of scattered X-rays in all directions. The scattered X-rays to be reduced are: those from above and from the sides for an additional shield box 1, which is a PD; and those from below for a high-performance table 2. Furthermore, by combining protective instruments in addition to PDs, it is possible to reduce the strength of scattered X-rays that leak to the sides from a pass-through port of a box due to human body movements during a surgery. Further using composite absorption material in various locations enables lineal energy absorption, with scattered X-rays being weakened. Thus, it is possible to reduce the radiation exposure of the patient and the medical worker and reduce the burden of protection for the medical worker.

### CROSS-REFERENCE TO RELATED APPLICATIONS

This Application claims the benefit of priority and is a Continuation application of the prior International Patent Application No. PCT/JP2023/003711, with an international filing date of Feb. 6, 2023, which designated the United States, and is related to the Japanese Patent Application No. 2022-018334, filed Feb. 8, 2022, Japanese Patent Application No. 2022-075633, filed May 1, 2022, Japanese Patent Application No. 2022-123002, filed Aug. 1, 2022, Japanese Patent Application No. 2022-161788, filed Oct. 6, 2022 and Japanese Patent Application No. 2022-205553, filed Dec. 22, 2022, the entire disclosures of all applications are expressly incorporated by reference in their entirety herein.

### TECHNICAL FIELD

The present invention provides a compounded protective device and instrument that reduces the exposure dose and radiation protection burden for medical workers and patients in the medical field that utilizes “X-ray fluoroscopy equipment”. This instrument is an add-on to a medical under-tube fluoroscopy.

### BACKGROUND OF THE INVENTION

The use of radiation in the medical field is becoming more widespread. A typical

example is treatment using interventional radiology (hereinafter referred to as “IVR”) technique. In IVR, a thin tube or needle called a catheter is inserted into the body while observing X-ray fluoroscopic images and Angio graphic images. This allows it possible to treat diseases without surgical operation and with as little scarring scars on the body as possible.

Today, X-ray fluoroscopes are used by specialists (hereinafter referred to as “surgeons”) in various medical departments who perform numerous operations as clinical procedures. The use of radiation in clinical practice is steadily increasing, and procedures involving exposure of patients and medical workers are frequently performed. Recently, techniques such as pulsed fluoroscopy and selective Angiography have advanced further. However, as opportunities for use increase, the exposure time due to X-ray irradiation tends to become longer, leading to an increase in medical exposure for patients and occupational exposure for medical workers. In addition, in some cases radiation protection measures are lagging behind, increasing the risk of radiation sickness for medical workers and patients.

In Angiography, an X-ray fluoroscopy device (hereinafter referred to as an “Angio graphic device”) is used. Angio graphic device is a method for observing the shape of the blood vessel itself by injecting a contrast medium into the blood vessel and photographing the flow with X-rays. By injecting a contrast medium that does not easily pass through X-rays into the target blood vessel and then taking an X-ray image, it is possible to clearly show the shape of the blood vessel in the part containing the contrast medium. In addition, with the Angio graphic device, fluoroscopy or observation is always performed during the surgery. Therefore, medical procedures that use Angio graphic device expose medical workers and patients to high doses of radiation. Some of the recent Angio graphic devices use X-ray pulse irradiation in order to reduce medical exposure and occupational exposure of medical personnel.

Here, the structure and usage of Angio graphic device and the behavior of X-rays will be explained as a representative example. A typical structure of an Angio graphic apparatus is to install an X-ray tube either above or below a bed (hereinafter referred to as “table”) on which a patient lies. In addition, the pair of X-ray receiver installed in opposite directions receives the X-rays that have passed through the patient. In recent years, lightweight flat panel detector (FPD) X-ray receivers that receive light with semiconductor sensors have been used. FPD is a flat panel complementary metal oxide semiconductor (CMOS) X-ray image sensor. Recently, FPDs have become capable of wirelessly transmitting continuously captured image information data. Therefore, FPD can be used independently.

In an Angio graphic device, the direct rays of X-rays inherently have a straight, unidirectional exposure path from an X-ray tube in the X-ray source, through the patient, to the image receiver. A small portion of the X-rays emitted from the X-ray tube and passed through the patient enters the image receiver. A portion of the incident X-rays is transmitted as fluoroscopic image data and displayed on a liquid crystal TV screen. However, most of the X-rays are scattered by the patient's body and emitted as scattered radiation around the apparatus.

In the case of an under-tube type Angio graphic device in which the X-ray source is placed below the table, the primary X-rays from the X-ray tube and their small angle scattered X-rays travel upward. However, the X-rays scattered by the table and the patient's body travel not only upward, but also to the sides and downward. Note that, according to Non-Patent Document 1 (ICRP, 2017), it is stated that in the under-tube type scattered X-ray, the number of photons is the largest below the table.

Patent Document 1 states that, for example, in the case of an under-tube type primary X-ray of about 100 KeV, the ratio of scattering and absorption is as follows. Approximately 80% is scattered by body tissues and 10% is absorbed. More is scattered by the table and about 3% is absorbed. This results in approximately 3% being transmitted to the X-ray receiver.

For radiological protection of medical personnel and patients, Non-Patent Document 1 (ICRP, 2017) proposes the use of protective equipment. For personal use, this protective equipment is a lead apron, eye protection (safety glasses), and thyroid protection. For multiple people, there are protective curtains, ceiling-suspended shields, and mounted shields. However, these radiation protection devices are designed to protect only from X-rays that come in a planar direction toward a specific direction, that is, from only the intended direction. Therefore, the effect of reducing exposure by this radiation protective equipment is limited to a specific direction. Therefore, the purpose of this protective equipment is to reduce to some extent the radiation exposure of medical personnel who move around during surgeries. This protective equipment does not provide a fundamental solution to reducing exposure to X-rays scattered from various directions.

In particular, in recent years, in order to reduce the radiation exposure of the operator's eyes, the crystalline lens dose is restricted by law. As a result, more and more IVR doctors are wearing crystalline lens dosimeters in addition to protective glasses. However, the small angle scattered X-rays from the periphery of the irradiation field in the under-tube type have high energy and have the same intensity as the primary X-rays. These forward scattered X-rays are directed to the operator's eyes. Therefore, current lead glasses, goggles, etc. do not adequately shield the bottom and sides of the glasses. However, protective glasses must be light in weight in order to reduce the physical burden. Therefore, there is a certain limit to sufficiently reducing the operator's lens dose.

As mentioned above, the scattered X-rays come from three directions: front, side, and rear when viewed from the patient's body. The energy of these scattered X-rays is roughly categorized into three types depending on the source and their effective energy. The highest energy is the small angle scattered X-rays that are generated upward (forward the under-tube) from the irradiation field and its surroundings. Small angle scattered X-rays are included in forward scattered X-rays. The next highest energy is scattered X-rays that are generated near the irradiation field in three directions: front, side, and rear (hereinafter referred to as “all directions”). The lowest energy is the scattered X-rays that are generated from the patient's whole body within 40 cm from the irradiation field and are re-scattered several times by human tissue (hereinafter referred to as “whole body scattered X-rays”).

Patent Document 1 relates to a composite absorption material for scattered X-rays by the same inventor. Patent Document 1 proposes a composite absorption material that attenuates scattered X-rays and absorbs them through “linear energy absorption” using a multilayer structure in which three or more layers having different roles are closely stacked. The composite absorption material is composed of a low reflection attenuation layer (initial layer) with an atomic number of 82 or more, and a multilayer absorption layer (diffuse absorber, electron absorber). In many cases, metals such as tin (Sn), niobium (Nb), molybdenum (Mo), copper (Cu), iron (Fe), titanium (Ti), and aluminum (Al) are used for multilayer absorption layers. Also, flat plates or foils of these metals are used. By arranging one to three pairs of diffusion absorbers and electron absorbers overlapping each other with no gaps, incident scattered X-rays are efficiently absorbed by “line energy absorption.” Secondary X-rays consisting of characteristic X-rays and brake X-rays are generated by “linear energy absorption”. The generated secondary X-rays are scattered in all directions including the direction of incidence. The scattered secondary X-rays travel toward the surrounding and side layer materials, where similar interactions occur. This is diffusion pushback. In all of these processes, the X-rays disappear by converting their energy into kinetic energy such as photoelectrons. This is electron absorption. The above-mentioned composite absorbing material attenuates the scattered X-rays generated in the patient's body during surgery, and then absorbs them through “linear energy absorption.”

In the background art of Patent Document 1, the results of investigation on the structure and materials of the prior art protective equipment are described. None of these armors had other materials comparable to the composite absorbent material of this invention. In other words, there isn't a material that attenuates scattered X-rays and absorbs them through “linear energy absorption” by laminating a low-reflection-attenuating layer and a multi-layer absorption layer in close contact with each other and using the outermost layer as an electron absorber with an atomic number of 11 to 30. Therefore, there is no material similar to the composite absorbent material of Patent Document 1.

Further, in Example 17 and FIG. 13 of Patent Document 1, a hanging cloth on a patient's body, a sheet on a table, clothes, and a head cover using a composite absorbent material are shown as examples. Scattered X-ray absorbers such as draperies, clothing and head coverings mainly use flexible composite absorbing materials. A table liner may use a rigid composite absorbent material. In addition, the above-mentioned hanging cloths and sheets are hollowed out for the radiation field. Note that Patent Document 1 is a material patent and does not claim protective gear or its structure.

Patent Document 2 is related to a medical table that transmits X-rays well and reduces scattering by the same inventor (hereinafter referred to as a “high-performance table”). This high-performance table consists of the following three stages at maximum. This consists of a top plate tier that supports the weight of the patient and absorbs scattered X-rays, an intermediate tier that serves as a movable diaphragm for the irradiation field and an absorber, and a bottom plate tier made of low-reflection and low-scattering material. A certain function can be achieved even with only one or two of these three stages. In this table, primary X-rays pass through a net or thin sheet (thin film) of carbon fiber reinforced plastic (hereinafter referred to as “CFRP”) installed on the tabletop. Since they pass through hollow spaces of the middle tier and the bottom plate tier, they reach the irradiation field of the affected part of the patient's body without interaction. Further, a shielding material or a composite absorbent material (hereinafter referred to as “functional material”) is placed on the upper surface of the tabletop. This reduces the intensity of scattered X-rays generated in the patient's body.

In the background art of Patent Document 2, firstly, the research result of the apparatus for sharpening the image quality of the prior art X-ray receiver was described. The present invention was compared with a focusing grid or cross-grid and a circular shading sizing device that sharpened the image quality of the investigated X-ray receivers. These prior arts do not have a mechanism for transmitting primary X-rays in the irradiation field or a diaphragm mechanism. Next, the results of research on the structure and materials of table accessories for radiation protection was described. The present invention was compared with the investigated X-ray protective plate, X-ray protective equipment. These prior art techniques are partial shields and do not have the function of absorbing them through “linear energy absorption” after attenuating the scattered X-rays generated by the patient's body using the material of the tabletop. Therefore, the same high-performance table as that of Patent Document 2 cannot be found.

Patent Document 3 relates to an additional shield box or the purpose of reducing radiation exposure and reducing protective load by the same inventor (hereinafter referred to as “additional box”). The configuration of the additional box of this invention is as follows. The configuration consists of a box body, a box top plate, a viewing window, a patient port, the shielding sheet for the patient port, a sleeve port, and the sleeve structure for the sleeve port.

The additional box of Patent Document 3 has the following two types depending on the structure of the X-ray receiver. These are a split box type box and a built-in FPD type box.

The additional box is assembled and placed on the table, surrounding the irradiation field of the patient's trunk, etc. This additional box does not have an opening that communicates with the external space in any three-dimensional direction when it is not operated. The patient's body passes through the additional box through patient ports provided at both ends in the direction of height (hereinafter referred to as “body axis direction”). The head and lower extremities are placed outside the box to avoid exposure. Here, the remaining space of the patient port is closed off with a flexible shielding sheet.

Furthermore, during surgery, medical personnel can view the inside of the box through a viewing window with shielding capabilities. At the same time, a medical procedure is performed here by inserting hands and arms through a flexible sleeve structure attached to the sleeve port. Sleeve structures also have shielding capabilities.

In the background art of Patent Document 3, the research results of prior art human body protection devices, radiation shield devices, and radiation protection cabins are described. These devices do not have a device for operating the inside or a mechanism to protect the operator's hands and arms. In addition to having an unshielded aperture, these do not have a rectangular parallelepiped box that three-dimensionally surrounds the irradiation field. There is no mechanism to protect the head from small angle scattered X-rays directed upward. Furthermore, these systems do not have the concept of installing an X-ray receiver inside a box. Furthermore, there is no idea of placing a composite absorbing material on the surface on which X-rays are incident. In addition, the radiation shielding device does not provide lateral shielding in the axial direction of the patient. The radiation protection cabin has no human body penetration ports and no shielding above the table. Therefore, the same additional shield box as disclosed in Patent Document 3 cannot be found.

In the above description, protective devices such as the high-performance table of Patent Document 2 and the additional shield box of Patent Document 3 have been described. To reduce scattered X-rays, the high-performance table is in charge of the bottom. Similarly, additional shield boxes are responsible for the top and sides. However, it is impossible in principle to shield scattered X-rays generated from a patient's body in all directions with one of these protective devices. That is, the intensity of scattered X-rays in all directions must be reduced by a combination of these. Note that the intensity of X-rays herein means the Riemann sum of the number of photons of X-rays in the target energy range.

Patent Document 4 relates to a complex protective device/instrument (PDITS) aimed at reducing radiation exposure and reducing protection load by the same inventor. Patent Document 4 is a combination of Patent Documents 1 to 3 and further developed. That is, by combining the high-performance table of Patent Document 2 with the additional shield box of Patent Document 3, the intensity of scattered X-rays generated from the patient's body in all directions is reduced. Furthermore, by adding the hanging cloth and patient's garment, it is possible to reduce the intensity of scattered X-rays leaking laterally from the through port of the additional shield box as the human body moves during surgery.

The present invention is a combination of Patent Documents 1 to 3 and Patent Document 4, which is a further development thereof. In Patent Documents 1 to 3, there was no prior art similar to each invention. Therefore, the subject matter of the present invention is not present in the prior art.

### PRIOR ART DOCUMENT

### Non-Patent Literature

[Non-patent literature 1] ICRP Publication 117, “Radiological Protection in Fluoroscopically Guided Procedures outside the Imaging Department.”, Ann ICRP 40 (6), 2010

### Patent Literature

[Patent literature 1] JPA 2022-161788 (Domestic priority application, earlier application JPA 2022-001336)
[Patent literature 2] JPA 2022-123002 (Domestic priority application, earlier application JPA 2022-018334)
[Patent literature 3] JPA 2022-075633
[Patent literature 4] JPA 2022-205553 (Domestic priority application, earlier applications are Patent Documents 1 to 3)

### SUMMARY OF THE INVENTION

### Problems to be Solved by the Invention

Conventional protective equipment can only protect against X-rays from specific directions. Additionally, conventional protective equipment imposes a physical burden on the medical personnel wearing it due to its mass, shape, and structure. Furthermore, in many cases, only specific organs of medical workers are directly protected.

Therefore, it is not possible to protect against X-rays in directions other than a specific direction, such as scattered X-rays generated from the patient's body in all directions. The present invention reduces the exposure dose of medical personnel due to scattered X-rays generated from a patient's body in all directions. Moreover, the present invention reduces the physical burden of radiation protection on medical personnel.

### Means to Solve Problems

The present invention consists of two types: a) a combination of a protective device (PD) and/or a protective instrument (PI) and a protective tool (PT), and b) a single unit protective device (PD) with many functions. Further, the PD in b) above is a part of the PD in a) above.

The first protection is a medical table as a PD. This table has a functional material placed on the upper surface of the table, which has a shielding ability against scattered X-rays from a patient placed thereon. The high-performance medical table of Patent Document 2, which is one of the first protections, transmits primary X-rays well and reduces scattering. This also reduces the intensity of downwardly directed scattered X-rays generated by the patient's body.

The second protection is a medical box as a PD. This box is placed above the table and surrounds the patient's body above and on the sides, with the functional material disposed on or inside the box where the scattered X-rays are incident. One of the second protections, the additional shield box of Patent Document 3, reduces the intensity of upwardly and laterally scattered X-rays generated from the patient's body.

By combining the first and second protections, which are PDs, primary X-rays can be transmitted well, and the intensity of scattered X-rays generated from the patient's body in all directions is reduced.

The third protection is the protective instrument (PI) of Patent Document 4. The PI includes patient hanging cloth, patient's clothing, and patient's head coverings. In addition to the first and/or second protection that is PD, PI is added as a third protection. The reason for this is to reduce the intensity of scattered X-rays leaking laterally from the penetration port of the additional shield box as the human body moves during surgery. The protective device (PD) of the present invention is a shielding glove or cuff for medical personnel that complements the shielding function of PDs and PIs. The concept is different from conventional protective aprons and protective glasses that directly protect specific organs of medical workers.

In the means for solving the problems of the present invention, a single protective device (PD) provided with many of the above b) functions will first be described so that a specific example can be shown. Next, PDITS (Protective devices, instruments, and tools) that combine PD and/or PI and PT in a) above at the beginning will be explained.

First, one type of device with many functions in the protective device (PD) of b) above at the beginning will be described. The first protective high-performance medical table has the function of providing good transmission of primary X-rays and reducing scattering. A mesh transmission plate unit is installed on the top plate level, and substances that interact with the primary X-rays are excluded from the lower middle level and the bottom plate level.

Generally, the role of a medical table is to support the weight of the patient's body. Therefore, the tabletop must support the mass of the patient's body at all points. Wire rod or net is preferably installed at a site that supports the patient's weight and allows primary X-rays to pass through. However, in the case of wires or nets with a large diameter, there is a possibility that the image of the X-ray receiver will have a thin white shadow like that of graph paper. Therefore, it is conceivable to install a strip-like film or a planar plate material (hereinafter referred to as “thin plate sheet”) such as CFRP or the like having a thin uniform thickness instead of the net. Wire rods, nets and thin sheets are preferably made of a single element or a compound of elements having an atomic number of 14 or less that hardly absorbs X-rays.

Example 7, which will be described later, describes the details of the high-performance table. Further details are described in Patent Document 2.

In addition, the high-performance medical table, one of the first protections, has the function of reducing the intensity of scattered X-rays downward from the patient's body. First, the intensity of scattered X-rays of low energy downward from the whole body can be reduced by the tabletop having the functional material placed on the surface facing the patient. Furthermore, the intensity of the scattered X-rays downward from the periphery of the irradiation field can be reduced by the middle tier slide table and aperture plate in which the functional material is placed on the surface facing the patient. Note that the backscattered X-rays of this region have higher energy than those of other regions.

The additional shield box (hereinafter referred to as “additional box”), which is one of the second protections, has the function of reducing the intensity of scattered X-rays upward and to the sides from the patient's body. The rectangular parallelepiped shape additional box has a structure in which functional materials are present in any three-dimensional direction. The intensity of side-scattered X-rays, which are of moderate energy, can be reduced by the functional material placed on the inner surface where the scattered X-rays are irradiated and the structural material of the box. There are two types of this additional box: a split box type and an FPD built-in type.

Example 8 below describes the details of the additional shield box. Further details are described in Patent Document 3.

When the energy of the small angle scattered X-rays coming from the periphery of the irradiation field is 88 KeV or higher, secondary X-rays containing the characteristic X-rays of the K shell are generated by shielding with Pb. Therefore, reflection and scattering of high X-ray energies occur. This is difficult to attenuate significantly with conventional box structural and shielding materials. In this case, as described in Patent Document 1 and Patent Document 3, this small angle scattered X-ray is attenuated by a box top plate to which a linear attenuation material such as uranium (U) with an atomic number of 83 or higher is added, and by a viewing window whose shielding ability is enhanced. Details of this are described in Patent Document 3.

Next, a composite protective device/instrument by combining the device, equipment, and protective tool mentioned in a) above at the beginning will be explained. These can reduce the intensity of scattered X-rays generated from the patient's body in all directions (front, side, and rear), reducing the exposure dose for medical workers and patients. At the same time, the burden of radiation protection on medical workers can be reduced. PDITS is a general term for protective devices, protective instruments, and protective tools. There are two main ways to solve problems:

The first means of combination uses two protective devices (PDs) in combination: the first protection, the table, and the second protection, the box. This allows primary X-rays to pass through well and reduces the intensity of scattered X-rays in all directions.

The second means of combination uses a protective instrument (PI) as the third protection, in addition to the above-mentioned protective device (PD). This reduces the intensity of scattered X-rays leaking laterally from the through port of the shielded box as the human body moves during surgery. The third type of protection, Protective Instrument (PI), includes hanging cloth over the patient's body, patient's garments, and head coverings. In addition, there are additional protective instrument (API) and simplified protective instrument (SEPI), which will be described later.

Conventional protective tools (PTs) such as protective clothing worn by medical workers are often limited to one specific direction and cannot be expected to provide major radiation protection. These PTs are protective aprons, protective eyewear, and thyroid protectors worn by medical workers. Therefore, in the present invention, PT is positioned as an additional means of PD and PI.

First, the types of scattered X-rays that are generated from the patient's body in all directions will be organized.

The average value of the energy of scattered X-rays generated in all directions from the patient's body is as follows. Here, the highest value is the forward scattered X-rays directed above the table. The next highest is the side scattered X-rays in the 360-degree (deg) direction of the horizontal plane on the side of the table. The lowest is the backscattered X-rays directed below the table.

Example 2 of Patent Document 1 and FIGS. 2B-1 to 2B-3 thereof describe the median energy of X-rays scattered by the patient's body in an Angio graphic device. This is 65 kiloelectron volts (KeV) on the side of a scattering angle of 90 degrees (deg) when the tube voltage of the X-ray source is 110 kilovolts (kV). Similarly, the back of a scattering angle of 150 degrees (deg) at 100 kV is 40 KeV.

On the other hand, there is literature information that reports a high value for the energy of forward scattered X-rays directed upward. This is natural since it includes small angle scattered X-rays of primary X-rays. However, the results were measured using an area dosimeter, and no literature could be found that accurately determined the measurement location.

Next, the materials used for protective devices and instrument (PDITS) will be explained. Here, existing materials containing elements such as lead (Pb), barium (Ba), or tungsten (W) and having shielding ability are referred to as “shielding materials.”

The material defined in Patent Document 1 that can effectively attenuate and absorb scattered X-rays by using a multilayer structure of three or more closely stacked layers is called a “composite absorption material.” The composite absorption material is composed of a low reflection attenuation layer (initial Pb layer) and a multilayer absorption layer.

Shielding materials such as Pb alone can greatly attenuate scattered X-rays. However, since the composite absorbing material has multiple absorbing layers, it attenuates the X-rays and absorbs them through “linear energy absorption” of most of the scattered X-rays below 87 KeV. Furthermore, in the present invention, both the above-mentioned shielding material and composite absorbent material having shielding ability are collectively referred to as “functional material.”

Further, the differences between the “table” and “high-performance table” and the “box” and “additional shield box” of the present invention are shown below.

The “table” of the present invention is a flat table on which a functional material capable of shielding against scattered X-rays from a patient placed thereon is disposed. The high-performance table takes care of the lower part in reducing the intensity of scattered X-rays. The table is one of the protective devices (PDs).

The “high-performance table” of the present invention increases the transmittance of primary X-rays and reduces scattered X-rays. The high-performance table has the above-mentioned table added with a function of suppressing the absorption and scattering of primary X-rays by the top plate and increasing the transmittance of primary X-rays. Furthermore, this table has an added function of reducing the intensity of scattered X-rays directed downward from the vicinity of the irradiation field of the patient's body. To give a more precise definition, the high-performance table has a net made of elements or compounds with an atomic number of 14 or less installed on the tier of the top plate. Due to this effect, the high-performance table is a table that suppresses absorption and scattering of X-rays by the top plate by allowing X-rays to pass through the mesh portion. The high-performance table is a narrow concept within a table that is the first protection and is one of the protective devices (PDs).

The “box” of the present invention is a rectangular box that is placed on a table and has a functional material arranged on the surface or inside of which the scattered X-rays are incident. The box shares the upper and side parts in reducing the intensity of the scattered X-rays. It is essential for the box of the present invention to incorporate the X-ray receiver into the box. Providing a patient port through the patient's body is optional. However, it is recommended that it be installed as an additional function in order to control medical radiation exposure to patients and to reduce the psychological burden on patients. The box is a type of protective device (PD).

The “additional shield box” of the present invention is a box-shaped additional shield that reduces radiation exposure and protection load for medical personnel. This box has the functions of a device for visually checking the inside of the box, such as a viewing window, and a device for operating the inside such as the sleeve port. The box top plate's radiation attenuating material attenuates and absorbs small angle scattered X-rays, which have a high intensity upward from near the irradiation field of the patient's body. Additional shielding box is a narrow concept within the box which is the second protection and is one of the protective devices (PDs).

Furthermore, the definition of “omnidirectional” in the present invention and the difference between “aperture” and “no aperture” are shown below.

In the present invention, “omnidirectional” means all directions of 360 degrees, including all directions, up, down, left, and right, which are equal to the direction of the radiation spreading from the radiation source located at the center of the sphere. In the present invention, when the vertically upper part of the sphere is 0 degrees (base point) and the vertically lower part is 180 degrees, the sphere with the center angle of 0 to 45 degrees and lateral 360 degrees is called upward. The sphere with the center angle of 135 to 180 degrees and lateral 360 degrees is called downward. The remaining sphere with the center angle of 45 to 135 degrees and lateral 360 degrees is called the sideward.

The term “opening” in the present invention refers to the mouth in an open state. Penetration ports that are blocked by a structure in which a functional material with shielding ability is disposed on or inside the structure are excluded from the list of openings, which are referred to as “openings in which transmission of X-rays cannot be reduced.” That is, in the present invention, “no opening” means a state surrounded in all directions by a structure in which a functional material with shielding ability is arranged on the surface or inside.

The first means of combination utilizes two protective devices (PDs), primary protection and secondary protection, in combination. The necessary condition of the first protection is “a table” topped with a functional material capable of shielding against scattered X-rays from a patient on top of it. If this table is a high-performance table to which primary X-ray transmission and aperture functions are added, it becomes a more preferable composite protective device (PD). The necessary condition of the second protective is “a box” surrounding the patient's body above and to the sides placed on the surface or inside where the scattered X-rays are incident. If this box is an additional shield box with additional functions such as internal visibility and human body penetration, it becomes a more preferable compound protective device (PD). A means of combining two protection devices, the first protection and the second protection, is called a “combination case” of a box and a table.

In the first method of combination, two protective devices (PDs) share the orientation from the patient's body to reduce the intensity of the scattered X-rays as follows. The table is in charge of the lower direction. The box is in charge of the upper and lateral directions. Combining these two PDs has the following two effects. One is that it transmits primary X-rays well. The other is to reduce the intensity of scattered X-rays generated from the patient's body in all directions, including the periphery of the irradiation field. A PD that combines multiple devices is called a “complex protective device.”

If the PD table is a high-performance table, the tube voltage of the X-ray source can be lowered by transmitting primary X-rays well. Lowering the tube voltage can reduce the intensity of the generated scattered X-rays. When the intensity of scattered X-rays is reduced, the exposure dose can be reduced. Therefore, these two are related.

Example 1 and FIGS. 1A to 1D explain in detail the concept of a combination case of a box and a table based on the first means. Example 2 and FIGS. 2A to 2B-3 illustrate a bird's eye view of the combination of two protective devices. FIGS. 1A to 1D and FIGS. 2A to 2B-3 use a high-performance table for the table and an additional shield box for the box.

The second method of combination, adds, in addition to the first protection (table) and second protection (box) of the first means, a protective instrument (PI) as a third protection. The reason for adding the third protection is to reduce the intensity of scattered X-rays leaking laterally from the through port of the shielded box as the human body moves during surgery. There are three ways to think about adding PI, and the means to achieve each are different. The PI is different from the PT worn by medical professionals in the past. All of these PIs are made of functional materials.

The 1'st of the second method of combination is the idea that protection from side scattered X-rays, which is insufficient with only a protective device (PD) during surgery, is supplemented by an additional protective instrument (PI) that is attached to the patient's body. Here, a hanging cloth (hereinafter referred to as “normal hanging cloth”) having a normal mass range and the patient's garments are used as the additional PI. If the neck or head is in the irradiation field, a head cover in addition to clothing is used.

The 2nd of the second method of combination uses a newly devised thick hanging cloth as PI. A support structure that supports this mass without placing any physical strain on the patient's body was also devised. The collective term for these is “Additional Protective Instruments” (API).

The 3rd of the second means of the combination is a modification of the 2nd. The protection from side scattered X-rays enhanced by API creates a margin for reducing the protection provided by the box attachment, which combines the sleeve structure and shielding sheet on the PD side. The idea is that this will improve the operability of the protective instrument (PI) attached to the PD while maintaining the same level of protective performance. This PI is the Strip-type curtain and gloveless port. The collective term for these is “Simple and Easy Protective Instrument” (SEPI).

The details of the second means 2, the second means 3, and the combination case in which they are evolved are explained in Patent Document 4.

At the outset of the description of the second method of combination, the condition of the box sides that may result in insufficient shielding during surgery is described. The rectangular parallelepiped shape additional shield box has a sleeve port and a patient port at the side ends. Both are collectively referred to as a “through port.”

A sleeve port is present on the side end face with a viewing window for a medical practitioner to insert hands and arms into the box during surgery. A flexible sleeve structure with a shielding function is attached to the sleeve port. The sleeve structure allows medical personnel to perform medical procedures within the box with reduced radiation exposure to their hands and arms. This sleeve structure may be a sheet with a shield or a strip-type curtain.

The patient port exists on the side end in the body axis direction for penetrating the patient's trunk during surgery. The patient's head and legs are exposed outside the box which has a shielding function. This can reduce medical radioactive exposure to patients and ease the psychological burden caused by confined spaces. The act of penetrating the patient's trunk through the patient port of the box is performed by a medical worker.

During observation such as before an operation (hereinafter referred to as “not in operation”), the penetration port is provided with a structure capable of shielding. In the shielding structure of Patent Document 2, the sleeve port has a sleeve structure. Similarly, the patient port has a shield sheet.

One typical example of a sleeve structure is a sleeve. The sleeve is a cone that is open at both ends and is made by molding a flexible functional material. The tip of the sleeve opens at the wrist size. When not in operation or not in use, it is closed with a rubber band. A medical worker wearing protective gloves inserts the arm during surgery. In this state, they use their hands and arms vigorously for medical procedures. Each time this movement occurs, the tip of the sleeve opens, and scattered X-rays leak out of the box. The shielding sheet of the sleeve port is a flexible sheet made of lead-containing resin or lead-containing rubber. One of these shielding sheets is a Strip-type curtain. Strip-type curtains are flexible curtains with shielding ability and many partitions. This partition consists of a large number of strip-shaped sheets with a small width in the horizontal direction and a large length in the vertical direction. A The strip-shaped sheets are suspended from the top of the through port. Since the strip-type curtain has a large number of flexible partitions, it can be easily opened by pushing with a part of the body. Accordingly, scattered X-rays easily leak to the sides.

On the other hand, the patient port shielding sheet is an integral sheet made of a flexible functional material. After installing the box through the patient in preparation for surgery, the shielding sheet is installed so as to close off the remaining space of the patient port as much as possible. This shields the patient port when it is not in operation. However, during surgery, the closed sheet may shift due to patient movement. If it deviates, scattered X-rays will leak out of the box.

Body tissue penetrating the penetration port partially scatters x-rays and partially transmits them. This body tissue is the hand of a medical worker or the trunk of a patient. That is, when the intensity of scattered X-rays inside the box is high, part of the scattered X-rays leaks out of the box.

In addition, soft and thin materials are used for sleeves and shielding sheets in order to carry out delicate medical procedures. That is, these flexible functional materials cannot be thickened due to medical requirements. Therefore, when the tube voltage of the X-ray source is high, the shielding capability of these box attachments may be insufficient.

As previously mentioned, the penetration port is shielded during not in operation before surgery. Efforts are made to close the gap as much as possible even when the body tissue is penetrated. However, during surgery, the movement of the penetrating body tissue creates a gap between the box and the human body, causing scattered X-rays to leak out of the box. In addition, X-rays pass through the human body tissue that has penetrated through the penetration port. Furthermore, when the tube voltage of the X-ray source is high, the shielding ability of the shielding structure may be insufficient. Therefore, through ports cannot completely prevent the leakage of X-rays during surgery.

In the 1st of the second method, the shielding ability insufficient in the protective device (PD) is supplemented by the additional protective instrument (PI) attached to the patient's body. The additional PIs in the box are the patient's hanging cloth and the patient's garment. These enhance the ability to shield scattered X-rays to the side of the box where the penetration port is located. Generally, the thickness of commercially available hanging cloth is 0.25-0.5 mm Pb in terms of lead equivalent. The clothing that are in the normal range of weights that are commercially available are referred to as “normal clothing.” Currently, there are no commercially available garments for this purpose.

In general, due to its shape effect, radiation can be shielded more efficiently with an article of the same thickness but with a smaller weight the closer it is to the radiation source. The hanging cloth on the patient's body and the patient's garment can shield the scattered X-rays emitted by the patient at optimal locations.

The hanging cloth is a flexible sheet made of functional material. In order to allow the primary X-rays to pass through, the hanging cloth is provided with a hollowed-out portion that cuts out the irradiation field of the affected area to a slightly larger size. The hollowed-out portion is open upward. Therefore, in the vicinity of the irradiation field, the ability of the hanging cloth to shield scattered X-rays upward is inferior to that toward the sides. Also, hanging cloth does not originally have the ability to shield downward.

The patient's garment should be worn by the patient, not by the HCW. The garment is in the form of a flexible short-hemmed cloak (eg half coat) made of functional material. The length of the hem is preferably within 40 cm from the edge of the radiation field. The patient's garment can block scattered X-rays emitted from the patient in all directions. However, clothes made from functional materials are heavy like armor. Therefore, it is difficult to use a large mass beyond the physical load limit of the patient's load. In addition, the patient's garment is provided with cut-out portions slightly larger than the irradiation field on the front and back for transmission of primary X-rays. Therefore, the ability of the patient's garment to shield scattered X-rays is inferior downward and upward compared to sideways.

The patient's head covering is a mask made of functional material. The patient is asked to wear it when the affected neck or head is the irradiation field. The reason for this is that scattered X-rays leak from the head to the outside when the distance from the irradiation field is within 40 cm.

As mentioned above, by combining multiple PDs with multiple PIs during surgery, it is possible to reduce the leakage of scattered X-rays, mainly to the sides, outside the box during surgery. Therefore, radiation exposure of medical personnel and patients can be reduced.

In Example 4 and FIGS. 4A to 4F, which will be described later, the detailed content of the concept of complex protective device, instruments, and tools (PDITS) that efficiently reduces exposure dose based on the 1st of the second method will be explained. In Example 5, a bird's-eye view of a case in which a new PI is combined will be explained with reference to FIG. 5. Further details are explained in Patent Document 4.

### Effect of the Invention

By combining a plurality of protective devices (PDs), the present invention can transmit primary X-rays well and reduce the intensity of scattered X-rays in all directions, including the periphery of the irradiation field. The scattered X-rays targeted for reduction are the top and sides of the PD box, and the bottom of the table. Among “the tables”, high-performance tables have additional functions such as transmission of primary X-rays and aperture function. Among “the boxes”, the additional shield box has additional functions such as the ability to see the inside and penetrate the human body. Additionally, by combining PD with protective instrument (PI: patient hanging cloths and patient's garment), it is possible to reduce the intensity of scattered X-rays that leak laterally from the box's penetration port as the human body moves during surgery.

These protective devices and instruments (PDITS) use composite absorbing materials to attenuate scattered X-rays and absorb them through “linear energy absorption.”. This reduces the radiation exposure of medical personnel and patients, and also reduces the protective burden on medical personnel.

### BRIEF DESCRIPTION OF DRAWINGS

FIGS. 1A to 1D are explanatory diagrams showing the method and effect of a combination case of the first protection (box) and the second protection (table). Here, FIGS. 1A and 1B are the case without protective device, FIGS. 1C and 1D indicate the “combined case” of the protective device (PD).

FIGS. 2A to 2B-3 are bird's-eye views of a “combination case” in which multiple protective devices (PDs) can reduce the intensity of scattered X-rays in all directions.

FIGS. 3A to 3E are explanatory diagrams of a new protective instrument (PI) added during surgery.

FIGS. 4A to 4F are explanatory diagrams showing the location of the through port of the box and the method and effect of reducing the intensity of X-rays scattered to the side during surgery. FIG. 4A to FIG. 4C-2 are the state before the countermeasure, FIG. 4D-1 to FIG. 4F are the state after taking measures.

FIG. 5 is a bird's-eye view of the “new PI combination case” with the addition of the third method of protection (protective instrument).

FIGS. 6A to 6C are bird's-eye views of the high-performance table disclosed in Patent Document 2, which transmits X-rays well and reduces scattering.

FIG. 7 is a bird's eye view of the split box type additional shield box disclosed in Patent Document 3.

FIGS. 8A to 8D are configuration diagrams of a basic case of the composite absorbent material of Patent Document 1.

FIGS. 9A to 9C are examples of the measurement results of the X-ray transmittance of the composite absorbent material of Patent Document 1 according to the JIS test.

### DETAILED DESCRIPTION OF THE INVENTION

Embodiments of the present invention will be described based on the drawings.

It should be noted that the protective devices and instruments (PDITS) and their constituent members shown here are merely examples and are not intended to limit the present invention.

In the remainder of this description, the following terminology will be used with respect to protective devices, protective instruments, and protective tools.

Unless otherwise specified, the position of the X-ray tube will be described using an under-tube type X-ray fluoroscope placed under the table as an example.

Boxes and tables are called protective devices (PDs). The drape over the patient's body, the sheet under the patient's body, the patient's garment, and the patient's head cover are called protective instruments (PIs). Protective aprons, protective goggles, protective gloves, and protective arm covers for medical workers are called protective tools (PTs). Collectively, protective device, protective instrument, and protective tool are referred to as PDITS. In addition, the general term for regular hanging cloth and thick hanging cloth is called hanging cloth.

The table is a table on which a functional material capable of shielding against scattered X-rays from the patient laid thereon is placed. The table is one of the protective devices (PDs).

The high-performance table is one of the tables and is one that transmits primary X-rays well and reduces scattering as shown in Patent Document 2. This is a table with two additional functions added to the above table. One is the function of increasing the transmittance of primary X-rays by suppressing absorption and scattering of primary X-rays by the top plate. The other function is to reduce the intensity of X-rays scattered downward from the vicinity of the irradiation field of the patient's body. A stricter definition is that a high-performance table has a net or thin plate made of elements or compounds with an atomic number of 14 or less installed on the top step, and X-rays pass through the net or thin plat, thereby this table suppresses the absorption and scattering of X-rays by the top plate. A high-performance table is a narrow concept within a table that is the first protection and is one of the protective devices (PDs).

The box is a box that surrounds the upper and lateral sides of the patient's body, with the functional material disposed on the surface or the inside onto which the scattered X-rays are incident. It is essential for the box of the present invention to incorporate the X-ray receiver into the box. Providing a patient port through the patient's body is optional. However, it is recommended that they be installed as an additional function in order to control medical radiation exposure to patients and to reduce the psychological burden on patients. A box is a type of protective device (PD).

The additional shield box is one of the boxes and is intended to reduce exposure and protect load as shown in Patent Document 3. The “additional shield box” of the present invention is a box-shaped additional shield that reduces radiation exposure and protection load for medical personnel. This box has the additional functions of a device for visually checking the inside of the box, such as a viewing window, and a device for operating the inside, such as a sleeve port. The box top plate's radiation attenuating material attenuates and absorbs small angle scattered X-rays, which have a high intensity upward from the vicinity of the irradiation field of the patient's body. The additional shield box is a narrow concept within the box that is the second protection and is one of the protective devices (PDs).

Furthermore, the definition of “omnidirectional” in the present invention and the difference between “aperture” and “no aperture” are shown below.

In the present invention, “omnidirectional” means all directions of 360 degrees, including all directions, up, down, left, and right, which are equal to the direction of the radiation spreading from the radiation source located at the center of the sphere. In the present invention, when the vertically upper part of the sphere is 0 degrees (base point) and the vertically lower part is 180 degrees, the sphere with a center angle of 0 to 45 degrees and an upper side of 360 degrees is called upward, and the sphere with a center angle of 135 to 180 degrees and a lower side of 360 degrees is called downward, and the remaining sphere with a center angle of 45 to 135 degrees and 360 degrees to the side is called the side.

The term “opening” in the present invention refers to the mouth in an open state. A through port that is blocked by a structure in which a functional material with shielding ability is disposed on the surface or inside is called an “opening in which transmission of X-rays cannot be reduced” and is excluded from the list of openings. In other words, “having no opening” in the present invention means a state in which the structure is surrounded in all directions by a structure in which a functional material with shielding ability is arranged on the surface or inside.

The diagnostic photography room, examination room, treatment room, and X-ray treatment room are collectively referred to as the “medical room, etc.”

Unless otherwise specified, a word described as an element means a material containing that element, and unless otherwise specified, a material refers to a material containing a single metal element.

Next, materials used for protective devices, instruments, and tools (PDITS) will be explained. Here, existing materials containing elements such as lead (Pb), barium (Ba), or tungsten (W) and having shielding ability are referred to as “shielding materials.”

A material defined in Patent Document 1 that can effectively attenuate and absorb scattered X-rays by using a multilayer structure of three or more closely stacked layers is called a “composite absorption material.” The composite absorbing material is composed of a low reflection attenuation layer (first layer Pb) and a multilayer absorbing layer.

Shielding materials such as Pb alone greatly attenuate scattered X-rays. However, since the composite absorption material has multiple absorption layers, in addition to attenuating X-rays, it also absorbs a large amount of linear energy of scattered X-rays of 87 KeV or less. Furthermore, in the present invention, both the above-mentioned shielding material and composite absorbent material having shielding ability are collectively referred to as “functional material.”

In the remainder of this description, the following terms will be used with respect to the types of X-rays.

The primary source of radiation is an X-ray tube, and this primary X-ray beam is called “primary X-ray.” The radiation that is scattered by the primary X-rays when they hit a patient/subject, a part of the device, etc. is collectively called “scattered X-rays.” The energy of X-rays means effective energy unless otherwise specified.

Among the scattered X-rays, the primary X-rays are scattered forward at a small angle while maintaining their energy. This is called “small angle scattering.” X-rays that have been scattered at small angles are called “small angle scattered X-rays.” Small angle scattered radiation occurs in the irradiation field and its surroundings. The periphery of the irradiation field refers to the area within 5 cm from the edge of the irradiation field, where forward scattered X-rays may be generated due to small angle scattering with one to several scattering times.

In addition, the primary X-rays are scattered by the patient's body, a table, etc., and are scattered forward at 0 to 45 degrees (deg) with respect to the incident angle. This is called “forward scattering.” X-rays that are forward scattered are called “forward scattered X-rays.” Similarly, it is scattered laterally between 45 and 135 degrees (deg). This is called “lateral scattering.” X-rays that are scattered sideways are called “side-scattered X-rays.” Similarly, it is scattered backwards between 135 and 180 degrees (deg). This is called “backscatter.” The X-rays that are backscattered are called “backscattered X-rays.” Note that small angle scattered X-rays are a type of forward-scattered X-rays and are one of them.

The embodiment of this description has the following configuration.

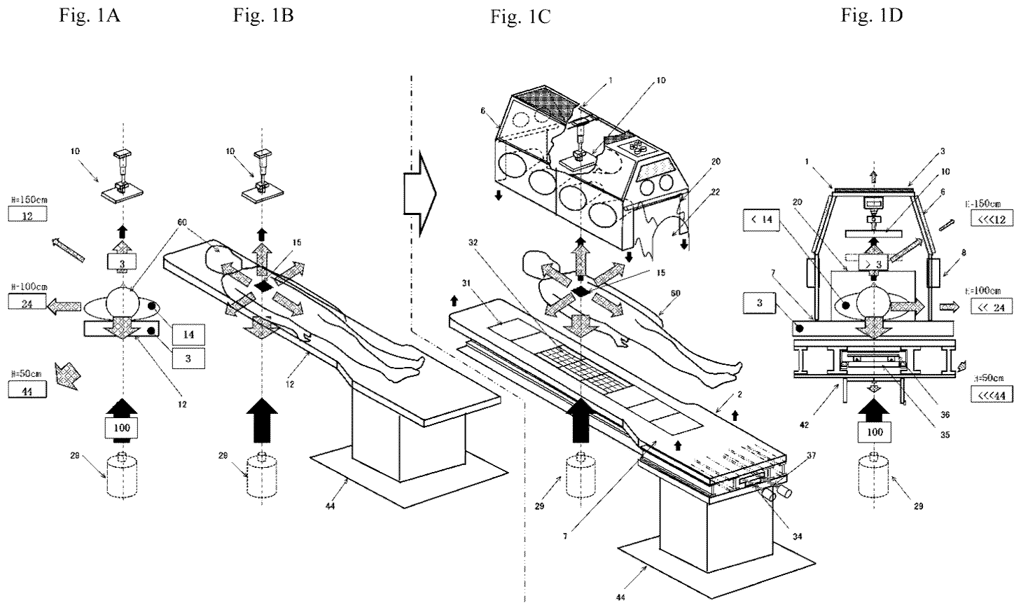

Example 1 explains the method and effect of a “combination case” in which the intensity of scattered X-rays in all directions can be reduced by combining a table and a box, which are protective devices (PDs), with reference to FIGS. 1A to 1D.

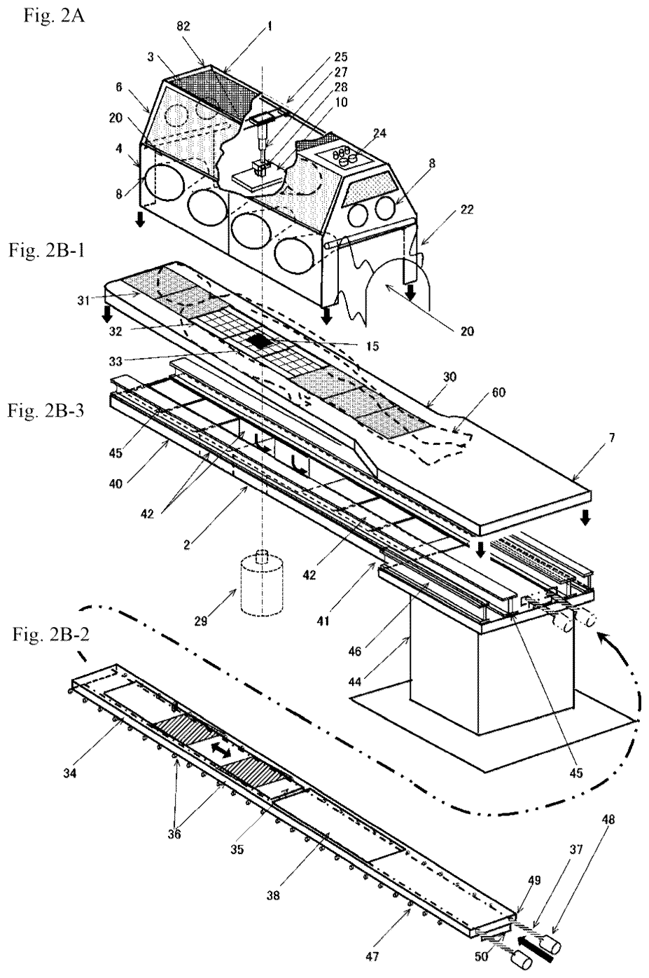

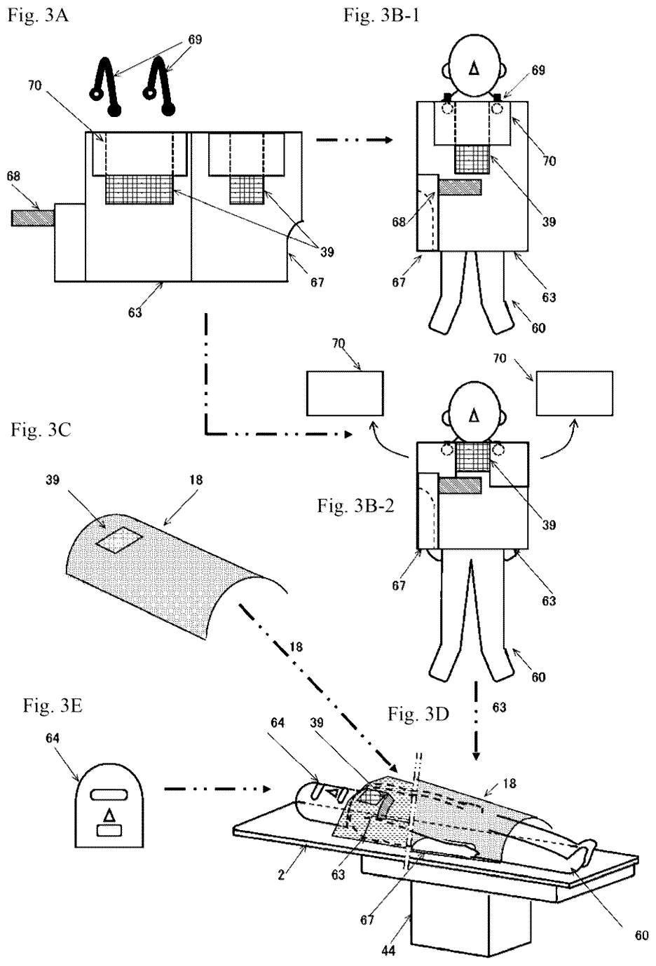

Example 2 explains a bird's-eye view of the “combination case” with reference to FIGS. 2A to 2B-3.

Example 3 explains a specific example of a new protective instrument (PI) added at the time of surgery with reference to FIGS. 3A to 3E.

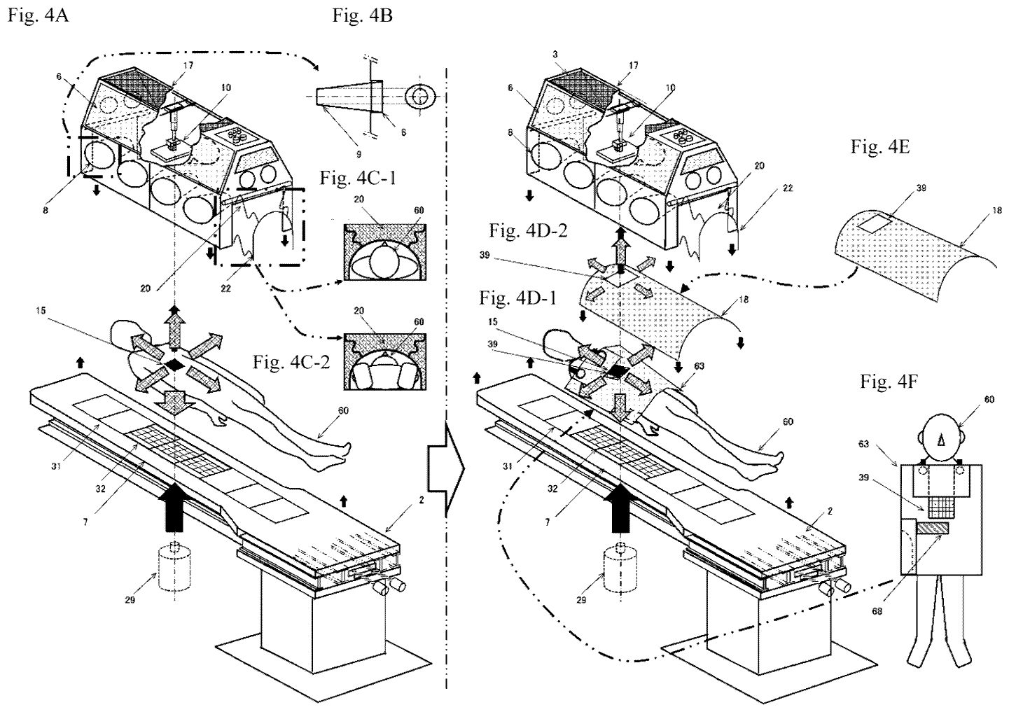

Example 4 explains a method and effect of reducing the intensity of X-rays scattered to the side during surgery with reference to FIGS. 4A to 4F.

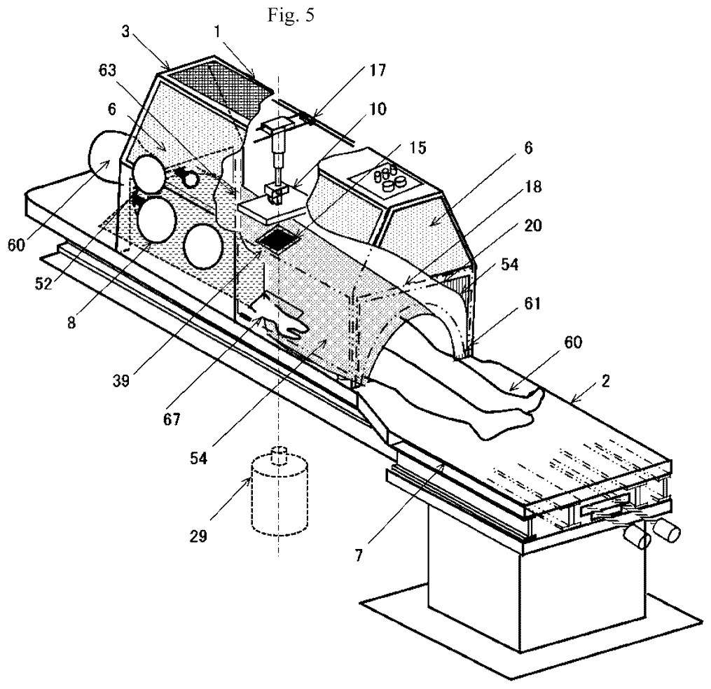

Example 5 explains a bird's-eye view of a case in which a new PI is combined as the third protection with reference to FIG. 5.

Example 6 explains a case in which the third protection is combined with either the first protection or the second protection.

Example 7 explains a high-performance table for medical use that transmits X-rays well and reduces scattering as disclosed in Patent Document 2.

Example 8 explains the additional shielding box of Patent Document 3 that reduces the exposure and protection load for medical personnel.

Example 9 explains the composite absorbent material of Patent Document 1.

Example 10 explains a part of the JIS test results of Patent Document 1.

### Example 1

### (Method and Effects of a “Combination Case” With a Table and a Box)

In Example 1, the method and effect of a “combination case” of a table and a box will be explained. In this case, two protective devices (PDs) are combined: the first protection, the table, and the second protection, the box. This makes it possible to reduce the intensity of scattered X-rays in all directions including the periphery of the irradiation field. FIGS. 1A to 1D are explanatory diagrams showing the method and effects of the “combination case”. The two-dot chain line and arrow in the center of FIGS. 1A to 1D distinguish between the conventional state (on the left) and the state after the present invention (on the right). FIG. 1A and FIG. 1B show the case where no protective device is present. FIG. 1C and FIG. 1D show the box and table combination case.

In FIGS. 1A to 1D, the directions of scattered X-rays are indicated by arrows. Roughly speaking, the thickness of the arrow indicates the number of photons of the scattered X-rays, and the length indicates the energy of the scattered X-rays. In this description, energy and the number of photons is collectively referred to as intensity.

FIG. 1A is described with reference to the numerical values of Patent Document 1. Here, assuming that the number of photons of the primary X-ray is “100”, the absorption by the body tissue (chest) is considered to be “14”. The absorption by the CFRP table is considered to be “3”. The transmission rate to the X-ray receiver is considered to be “3”. The remaining 80 is the scattering amount, which is considered to be “12” at a height of 150 cm (above), “24” at a height of 100 cm (sideways), and “44” at a height of 50 cm (downward). That is, backscattered X-rays directed downward have a large number of photons. However, the energy of X-rays is expected to be small. Note that this value is a guideline.

The effect of the box and table combination is to reduce the intensity of the scattered X-rays outside the box in FIG. 1D. FIG. 1D shows the degree of effectiveness by adding a less-than sign “<” to the numerical values in FIG. 1A, which shows the case without shielding or protective device. In other words, the upper (<<<12) and lower (<<<44) are expressed using three less-than inequality signs. The side (<<24) is expressed by two less-than inequality signs. For reasons described later, the scattered X-rays emitted to the sides are reduced to a slightly smaller extent.

In FIG. 1A and FIG. 1B, where no protective device is present, the downwardly directed backscattered X-rays have low energy but a high number of photons. Forward scattered X-rays that travel upward have higher energy than those directed downward but have a smaller number of photons. The solid black line indicates the primary X-ray.

FIG. 1C and FIG. 1D show a combined case with an additional shielding box 1 and a high-performance table 2, where protective device is present. In FIG. 1C, the additional shield box 1 and the high-performance table 2 are shown separated into upper and lower parts in order to make it easier to see the vicinity of the patient's body 60.

As indicated by the small black arrows pointing up and down, the table and box are in close contact with the patient's body in between.

The scattered X-ray arrows in FIG. 1B and FIG. 1C are the same because they are both inside boxes. Furthermore, the arrows of scattered X-rays are the same in the box in FIG. 1D and in FIG. 1C. Looking at the scattered X-ray arrows in FIG. 1D, the effect of the presence or absence of protective device inside and outside the box can be seen. Compared to both, the thickness and length are smaller outside the box in FIG. 1D.

As shown in FIG. 1D, the width and length of the upward and lateral arrows outside the box are reduced by the additional shielding box 1. That is, the intensity of emitted scattered X-rays is reduced. The intensity of the scattered X-rays emitted downward is reduced by the high-performance table 2. In particular, the width and length of the upward and downward arrows outside the box are significantly smaller. This is because the upper box top and viewing window and the lower tabletop have a high shielding ability. That is, the intensity of scattered X-rays emitted above and below the patient's body is significantly reduced by the protective device.

First, the effect of reducing the intensity of scattered X-rays will be explained. The degree of reduction is determined by the specifications (composition, material, and thickness) of the functional material of the protective device. The knowledge related to this is explained by the experimental results comparing the shielding materials (comparative Pb plates) of Examples 21 to 23 of Patent Document 1 and the composite absorbent material. This experiment is a transmission X-ray experiment (hereinafter referred to as “JIS test”) conducted in the present invention in accordance with the test criteria of the JIS standard. In this description, a part of this JIS test result is shown in Example 10.

The X-ray transmittance of the area where the composite absorbing material with the total thickness of the functional material (total-t)=0.4 mm to 0.5 mm is installed on the incident side surface can be evaluated from the above JIS test results. According to Example 10 of the present description, the transmittance of X-rays is approximately 1/50th when the tube voltage is 70 kV compared to the case where no shielding or protective device/protective instrument is present. Similarly, when the tube voltage is 50 kV, it is less than 1/200. On the other hand, when the tube voltage is 70 kV, the X-ray transmittance becomes about 1/2.5 when using only a Pb shielding material (comparative Pb plate) with a thickness of 0.2 mm. Similarly, when the tube voltage is 50 kV, it becomes about 1/9.5.

First, a functional material is placed on the surface of the structural material of the box on the incident side of the scattered X-rays. The shape of the box is often a long or short rectangular parallelepiped shape. Hereinafter, this will be referred to as a “rectangular parallelepiped box.” The structural material for the box is selected to have low mass and high strength. Examples of this structural material are aluminum alloys, titanium alloys or high strength plastics. The transparent lead-containing acrylic resin used for the viewing window 6 may constitute an integral box cover in the shape of a rectangular parallelepiped. This may be a split type instead of an integrated type. Furthermore, the shape of the box cover molded from the transparent acrylic resin may not be a rectangular parallelepiped shape but may be a semi-cylindrical shape. If a transparent sheet-like composite absorbing material 72 is placed on these surfaces, the intensity of scattered X-rays transmitted to the sides of the box will be significantly reduced, as shown in the JIS test results. Further, the intensity of the forward scattered X-rays, which are transmitted upward except for the periphery of the irradiation field, is significantly reduced. That is, by arranging the composite absorbing material on the inside surface of the box, the intensity of scattered X-rays transmitted upwardly and laterally is significantly lower than in the case without the aforementioned shielding or protective device.

Forward scattered X-rays including small angle scattered X-rays coming from the periphery of the irradiation field have high energy. Particularly when the energy exceeds 88 KeV and the low reflection attenuation layer is made of Pb, secondary X-rays containing the characteristic X-rays of the K shell are generated, resulting in increased reflection and scattering. In other words, linear attenuation is possible, but it cannot be effectively attenuated. As a countermeasure, a line-attenuating material such as bismuth (Bi) or uranium (U) is placed on the surface of the widened box top plate 7 on the incident side of the high-energy, forward-scattered X-rays. Linear attenuating materials have an atomic number of 83 or higher. Thereby, the low reflection attenuation layer can attenuate X-rays with low reflection and scattering. A multilayer absorber is placed on the side opposite to the surface of the low reflection attenuation layer having an atomic number of 83 or more that receives X-ray irradiation. This idea was presented by the inventor in the same patent document 3 as an expanded composite absorbent material. On the other hand, by increasing the angle of incidence of the viewing window with respect to forward scattered X-rays, a greater shielding ability can be obtained. When the energy is high, the above-mentioned treatment allows the box to attenuate the forward scattered X-rays and absorb them through “linear energy absorption”.

Next, a functional material is placed on the top surface of the structural material of the tabletop plate 7 of the table. This structural material is high strength plastic such as CFRP or aluminum alloy. In the case of the high-performance table 2, a functional material or a composite absorbent material 72 is arranged not only on the tabletop plate 7 but also on the upper surface of the absorption plate 31 and the aperture plate 36.

If a sheet-like composite absorbing material 72 is placed on these surfaces, the intensity of scattered X-rays transmitted below the table will be significantly reduced, as shown in the JIS test results. Furthermore, the intensity of backscattered X-rays transmitted downward from the periphery of the irradiation field is reduced by the functions of the slide table 35 and the aperture plate 36.

That is, if the composite absorbing material 72 is placed on the upper surface of the high-performance table 2 at various locations, the intensity of the scattered X-rays transmitted downward becomes smaller than that in the case without the above-mentioned shielding or protective device.

Therefore, in the case of a combination of a box and a table, the intensity of scattered X-rays emitted to the outside in all directions (three directions: upper, side, and lower) can be reduced. This is the effect when two protective devices (PDs), the first protection and the second protection, are combined during non-operation. That is, the first protection is a table on which a functional material capable of shielding against scattered X-rays from the patient placed on it. The second protection is a box that surrounds the patient's body above and to the sides, with functional materials placed on or in the surface onto which the scattered X-rays are incident.

Here, the first protection is the high-performance table 2, the second protection is the additional shield box 1, and if these are combined to form a composite protective device (PD), the intensity of scattered X-rays can be further reduced.

Next, the effect of the high-performance table 2 on transmitting primary X-rays well will be explained. In the high-performance table 2, most of the primary X-rays are transmitted through free space. That is, since the X-rays are not scattered by an object, there are fewer scattered X-rays generated from the primary X-rays. Further, this table can transmit primary X-rays with high positional accuracy. In the present invention, this phenomenon caused by the high-performance table 2 is referred to as “transmitting primary X-rays well.” When the primary X-rays are transmitted well, the image quality of the X-ray receiver becomes clearer. If the image quality of the X-ray receiver is sharp, the X-ray fluoroscope can be used with low primary X-ray energy. If the energy of the primary X-rays is lowered, the intensity of scattered X-rays generated from the patient's body can be further reduced. This synergistic effect can further reduce exposure to scattered X-rays.

In the combined case, this high-transmission feature of the high-performance table 2 increases the transmission rate of the primary X-rays to the X-ray receiver 10.

This can be seen by comparing the black arrow of the primary X-ray in FIGS. 1A to 1D with FIG. 1B and FIG. 1C.

In the present invention, by combining two protective devices (PDs: box and table), it is possible to transmit primary X-rays well and reduce the intensity of scattered X-rays in all directions.

### Example 2

### (Explanation of Bird's Eye View of a “Combination Case”)

In Example 2, a bird's-eye view of a “combination case” in which the intensity of scattered X-rays in all directions can be reduced by combining two protective devices (PD) will be described.

FIGS. 2A to 2B-3 show a bird's eye view of a combination case of a box and a table. FIG. 2A shows a built-in FPD type box 17, and FIGS. 2B-1 to 2B-3 show the high-performance table 2. Further, FIG. 2B-1 shows the top plate tier 30, FIG. 2B-2 shows the middle tier 34, and FIG. 2B-3 shows the bottom plate tier 40.

In FIGS. 2A to 2B-3, the high-performance table 2 and the additional shield box 1 are shown disassembled and divided into upper and lower parts so that the vicinity of the patient's body 60 can be seen. In reality, as shown by the small black arrows in the vertical direction, the table and box are in close contact with the patient's body in between. Also, the middle tier 34 of FIG. 2B-2 is removed and shown in the bottom position of FIGS. 2A to 2B-3. It should be noted that the middle tier 34 is actually a removable structure. Both the slide table 35 and the aperture plate 36 in FIG. 2B-2 move in the body axis direction.

The box, which is the second protection of the present invention, has a functional material arranged so as to three-dimensionally surround the irradiation field 15 corresponding to the affected area of the patient. The surface of each member constituting the box on the X-ray incident side is coated with a functional material. Among the boxes serving as the second protection, in the additional shield box 1, the operator can view the inside from the outside space through the viewing window 6, which is a resin or glass plate with shielding ability. The operator can operate by inserting the hands and arms through the sleeve structure 9 with shielding ability while visually checking the inside. Also, except for the patient port 20 and sleeve port 8, there are no openings that communicate with the external space.

The sleeve structure 9 of the additional box 1 is made of a flexible functional material such as a lead-containing arm sleeve. Lead-containing arm sleeves, etc. are used by attaching them to sleeve port 8.

The patient passes through the box axially through the patient port 20. This places the patient's head and body parts in the external space. Further, a flexible shielding sheet 22 with shielding ability attached to the patient port 20 closes the opening between the box body 4 and the human body. The excess shielding sheet 22 is rolled up by the holder 23. Power, electrical signals, optical signals and liquid communicate inside and outside the box through the box without openings by means of the shielding-capable connection connector 24.

Forward scattered X-rays include small angle scattered X-rays directed upward from the irradiation field of the patient's body and its surroundings, so the energy of the X-rays is high. If necessary, in this additional box, a line-attenuating material 82 with excellent shielding ability is further attached to the box top plate 3 to shield forward scattered X-rays directed upward. X-rays scattered to the side from the irradiation field and other parts are shielded by a box body 4 and a viewing window 6 having shielding ability. Furthermore, when the device is not operated, the penetration port is shielded by the sleeve structure 9 and the shielding sheet 22.

Among the tables that are the first protection of the present invention, the high-performance table 2 is composed of a maximum of three stages: a top plate tier 30, a middle tier 34, and a bottom plate tier 40. A certain level of performance can be achieved even with only one or two stages.

In FIGS. 2A to 2B-3, the support rails 45 and reinforcing beams 46 are not shown on the top plate tier 30 for easy visualization. Here, the bottom plate tier 40 is shown together with a table support 44. The middle tier 34 is accommodated by sliding into the support rail 45.

The top plate tier 30 of the high-performance table 2 has the following configuration. These are the tabletop plate 7, the absorption plate 31, the transmission plate unit 32, the support rail 45, and the reinforcing beam 46. The tabletop plate 7 on which the patient lies supports the weight of the patient's body. At the center of the axis of the tabletop plate 7, there is an opening that is long in the body axis direction. The absorption plate 31 is fitted and installed on the support rail 45 below the opening. The absorption plate 31 at a position that may become an irradiation field is removed, and the transmission plate unit 32 is installed. The mesh surface of the transmission plate unit 32 is made of a linear material such as CFRP or Al-based material that has high strength and is difficult to absorb X-rays. In some cases, a thin film made of CFRP is used instead of the mesh surface.

In the case of the under-tube type, the upper surfaces of the tabletop plate 7 and the absorption plate 31 are covered with a functional material. The aperture size of the irradiation field in the body axis direction can be adjusted using a slide table 35 and an aperture plate 36, which will be described later. The aperture size in the direction perpendicular to the body axis direction can be adjusted using an opening/closing plate 42 and a spacer 33 of the transmission plate unit 32, which will be described later. The load supported by the tabletop plate 7 is supported by the support rail 45 and the reinforcing beam 46, and finally by the table support stand 44.

The middle tier 34 of the high-performance table 2 is composed of a slide table 35, a aperture plate 36, a slide absorption plate 38, and a drive mechanism thereof. The slide table 35 is a flat plate that is long in the axial direction. There is an opening at a part of the center of the axis, and a slide absorption plate 38 is easily fixed onto the other part. A drive mechanism using a ball screw 37 for the aperture plate 36 is installed inside the slide table 35. A pair (two) of aperture plates 36 can be slid over the opening of the slide table 35 to freely adjust the size of the opening in the axial direction. The slide table 35 installed on the side rollers slides and moves in the axial direction, allowing the position of the opening to be adjusted freely. In this way, the position and size of the aperture in the axial direction of the irradiation field can be adjusted using the slide table 35 and the aperture plate 36.

The bottom plate tier 40 of the high-performance table 2 is composed of a bottom plate 41, an opening/closing plate 42, and a fixing/driving mechanism thereof. The opening/closing plate 42 can be opened/closed by controlling the opening angle using a hinge mechanism or the like. During surgery, the opening/closing plate 42 at the irradiation field position is opened. A mechanical device may be attached to the slide table 35, the aperture plate 36, and the opening/closing plate 42.

In the case of the under-tube type, the transmission rate of primary X-rays to the X-ray receiver 10 increases due to the ability of the high-performance table 2 to transmit X-rays well.

Furthermore, in the case of the under-tube type, the high-performance table 2 can reduce the intensity of backscattered X-rays directed downward from the irradiation field 15 of the patient's body and its surroundings by the tabletop plate 7 made of a functional material. Functional materials have shielding properties.

The upper side of the aperture plate 36 is covered with a composite absorbent material 72, and the lower side is covered with a shielding material 81. The slide absorption plate 38 is simply attached and installed at a location other than the opening position of the slide table 35, and the upper side is covered with a composite absorption material 72. Thereby, backscattered X-rays directed downward can be attenuated and then absorbed by “line energy absorption”.

The surface of the opening/closing plate 42 is covered with a shielding material 81 on the outside and a composite absorbent material 72 on the inside. Thereby, backscattered X-rays directed downward can be attenuated and then absorbed by “line energy absorption”.

In Examples 1 and 2, the method and effects of the “combination case” of boxes and tables were explained. By combining two protective devices (PDs), it is possible to reduce the intensity of scattered X-rays emitted from the patient's body in all directions (in three directions: upward, lateral, and downward), including the periphery of the irradiation field.

Here, if the first protection table is the high-performance table 2 and the second protection box is the additional shield box 1, the effect of reducing the intensity of scattered X-rays in all directions will be greater. In addition, a high-performance table is highly transparent to primary X-rays.

As a result, the air dose rate in medical rooms and the like can be reduced. Therefore, the radiation exposure of medical workers and patients can be reduced. Additionally, the protective burden on medical personnel can be reduced.

### Example 3

(Explanation of Specific Examples of New Protective Instrument (PI) Added During Surgery)

In Example 3, a specific example of a new protective instrument (PI) added as the third protection will be described. The new PIs described here are the hanging cloth that hangs over the patient's body and the patient's garment. In general, radiation can be shielded by a material with the same thickness but smaller mass as it approaches the radiation source due to its shape effect. Hereinafter, this will be referred to as the “shape effect of the shielding body.” The patient's body is the source of scattered X-rays. Therefore, the position of the hanging cloth and patient's garment has a large effect on reducing the intensity of scattered X-rays leaking out of the box even if the shield has a small mass.

The patient is asked to wear a patient's garment made of flexible functional material. Using the patient's garment as a shield is an unconventional idea. Moreover, as mentioned above, clothing can efficiently shield with a small mass due to the shape effect of the shielding body.

FIGS. 3A to 3E show explanatory diagrams of a protective device to be worn on a patient's body. FIG. 3A shows a developed drawing of the patient's garment 63. FIGS. 3B-1 and 3B-2 show the patient wearing the patient's garment 63. FIG. 3C shows a normal hanging cloth 18. FIG. 3D shows an installation diagram in which a head cover 64, patient's garment 63, and a normal hanging cloth 18 are used. FIG. 3E shows the head cover 64.

FIG. 3A is an example of a developed view of the garment 63 before being put on. The patient's garment 63 is often made of a sheet of flexible functional material. Therefore, sewing complicated shapes is avoided. In addition, both the front side and the back side are shaped mainly in straight lines. On the front side and the back side, there are cut-out portions 39 for the irradiation field through which the primary X-rays are transmitted. The width of the hollowed-out portion 39 on the back side is large. The patient's garment 63 has a front part, a back part, and a hand take-out port 67 that are continuous in one piece.

On the patient's garment 63, the position of the irradiation field 15 changes to the top, bottom, left and right of the human body trunk depending on the position of the affected area. Therefore, the patient's garment 63 can be folded or the wearing position can be shifted. In addition, the size is cut to be larger than the trunk of the human body. The vertical width of the hollowed-out portion 39 reaches the upper end of the patient's garment 63. The vertical width of the irradiation field is adjusted to the required size using the cover 70. The patient's garment 63 has a hand take-out port 67 so that the catheter can be easily inserted into the patient's wrist or arm during a procedure. Further, the patient's garment 63 can be suspended from the patient's shoulders using two shoulder straps 69. The length of the shoulder strap 69 can be adjusted. The attachment positions of the stoppers 68 on the cover 70, the hand take-out port 67, and the shoulder straps 69 can be easily changed because they are attached with hook-and-loop fasteners or cloth tape.

FIGS. 3B-1 and 3B-2 show the patient wearing the patient's garment 63. It is preferable that the patient's garment 63 can cover an area within 40 cm from the edge of the irradiation field 15.

FIG. 3B-1 shows how to wear the patient's garment 63 when the trunk of the body becomes the irradiation field 15. There is no need to bend the patient's garment 63 when performing X-ray fluoroscopy of organs in the trunk. When performing X-ray fluoroscopy on the abdomen below the stomach, the two shoulder straps 69 are lengthened and the patient's garment 63 is shifted downward when worn.

FIG. 3B-2 shows how to wear the patient's garment 63 when the neck becomes the irradiation field 15. The patient's garment 63 are pulled up and worn to perform X-ray examination of the neck. For this purpose, the upper end of the patient's garment 63 is bent. Also, the front and rear covers 70 is removed. The length of the two shoulder straps 69 is shortened.

When puncturing from the wrist in a catheter technique, the wrist and elbow of the patient's right hand are taken out from the hand take-out port 67.

After the hand has been taken out, the hand take-out port 67 can be secured to patient's garment 63 with a hook-and-loop fastener using a stopper 68.

Since the patient's garment 63 is worn on the patient, the patient must carry it by himself/herself. Considering the physical burden of the patient's weight, the mass of the patient's garment 63 cannot be made too large. Due to the weight limit that ordinary people can carry, the weight of patient's garment 63 must be at most 20 kg or less. More preferably it should be 15 kg or less, even more preferably 10 kg or less. Therefore, it is difficult to increase the thickness of the material that has the shielding function of patient's garment 63 due to mass limitations. If the mass of the patient's garment 63 is large, shorten the length of the hem so that it is 20 cm or more from the edge of the irradiation field 15.

FIG. 3C shows a normal hanging cloth 18 that is placed over a patient's body. This is a flexible sheet made of functional material. These hanging cloths have a cutout 39 in the area of the irradiation field 15. These hangings have a functional material encapsulated or placed on the surface on the side of incidence of scattered X-rays. The structure may be made of functional materials as is. Some have a laminated structure in which the surface is treated with resin or the like or laminated.

These hanging cloths on the patient's body can shield scattered X-rays emitted upwardly and laterally from the patient in optimal locations. These hanging cloths cannot shield the downward scattered X-rays. Furthermore, because of the hollowed-out portion 39, forward scattered X-rays directed upward from the irradiation field 15 and its surroundings cannot be blocked. Most of the X-rays scattered to the side can be shielded because they pass through the patient's body 60 and these hanging cloths.

Example 14 of Patent Document 4 shows a range in which the exposure dose within a patient's body increases during IVR. Here, the absorbed dose rate of scattered X-rays generated from the whole body of the patient is 3 to 4 mGy/min within 20 cm from the edge of the irradiation field. It was found that the range within 40 cm was approximately 0.2 mGy/min. That is, significant scattered X-rays are generated within 40 cm from the edge of the irradiation field. It is preferable that the area within 40 cm from the edge of the irradiation field 15 be covered with a normal hanging cloth 18. Further, it is preferable to cover the area within 20 cm with a thick hanging cloth 19 of 1 to 3 mm Pb.

In the case of a commercially available hanging cloth, the length may be longer. An example of a commonly available hanging cloth product is SGB manufactured by Hoshina Seisakusho. Its dimensions are 60 cm wide×100 cm long. Its mass is 3.2 kg with a lead equivalent of 0.25 mm Pb. Similarly, 0.5 mm Pb weighs 5.5 kg.

From the viewpoint of limiting the physical load due to the weight of the human body, the total mass of the normal hanging cloth 18 and patient's garment 63 must be at most 20 kg or less. Furthermore, the weight should preferably be 15 kg or less, more preferably 10 kg or less.

FIG. 3D is an installation diagram when a patient's garment 63 and a normal hanging cloth 18 are used. This figure uses the patient's garment 63 shown in FIG. 3B-2. Since this is a case where the affected area is the neck or the like, a head cover 64 shown in FIG. 3E is attached in FIG. 3D. A normal hanging cloth 18 has a length of 100 cm in the body axis direction. In FIG. 3D, the normal hanging cloth 18 has the largest length. Cloths 63 is 80 cm long.

FIG. 3E is a head cover 64. The head cover 64 is used when the affected neck becomes the irradiation field 15 and the head is within 40 cm from the irradiation field. In other words, this is a modification in which the neck of the patient's garment 63 becomes the irradiation field.

When the distance from the irradiation field 15 is short, scattered X-rays that have been scattered multiple times within the patient's body from the head leak into the outside space, increasing the air dose rate. This exposes medical workers to radiation. To reduce radiation exposure for medical personnel, the patient is fitted with a head cover 64 made of a flexible functional material or composite absorbent material.

The head cover 64 is a mask-like headgear, in which the eyes, nose, mouth, etc. are cut out and exposed, and the other parts are covered with a flexible functional material or a composite absorbent material. Thereby, emission of scattered X-rays from the head to the outside can be reduced.

Note that when the head becomes the irradiation field 15, the head must necessarily be housed in an additional box that houses the X-ray receiver 10.

Normal hanging cloth and patient's garments can shield scattered X-rays generated in the patient's body with a small mass due to the shape effect of the shielding body described above. The combination of these two PIs can reduce the intensity of scattered X-rays. However, since there is a hollowed-out portion that allows primary X-rays to pass through, the intensity of scattered X-rays upward and downward from the periphery of the irradiation field cannot be reduced. The role of the box described above is to shield forward scattered X-rays directed upward from the periphery of the irradiation field. Similarly, the role of the table mentioned above is to shield downwardly directed backscattered X-rays.

The patient's body bears the load of a patient's garment and normal hanging cloth. In order to limit the physical load on the human body, the total mass must be at most 20 kg or less. More preferably 10 kg or less.

By combining a new protective instrument (PI) with a composite protective device (PD), it is possible to reduce the intensity of scattered X-rays inside the box. In particular, it has the meaning of reducing the intensity of scattered X-rays leaking to the sides during surgery, which will be described later.

### Example 4

### (Method and Effect of Reducing the Intensity of X-Rays Scattered to the Side During Surgery)

In Example 4, a method and effect of adding a plurality of new protective instrument (PI) to the two protective devices (PDs) shown in Example 1 and Example 2 will be explained. The new PI for Example 4 is the patient's normal hanging cloth and patient's garment. The table is the first protection, the box is the second protection, and the new PI is the third protection. By adding a new PI, it is possible to reduce the intensity of scattered X-rays leaking to the side outside of the box during surgery.

FIGS. 4A to 4F are explanatory diagrams showing a method and effect of reducing the intensity of sideward scattered X-rays when the penetration port portion of the additional box 1 and a plurality of PIs are added. The two-dot chain line and arrow in the center of FIGS. 4A to 4F distinguish the state before the countermeasure (on the left) and after the countermeasure (on the right). FIG. 4A to FIG. 4C-2 show the state before the countermeasure, and FIG. 4D-1 to FIG. 4F show the state after the countermeasure. The two-dot chain line and arrow in the middle of FIGS. 4A to 4F distinguish the state before and after the countermeasure. FIG. 4A and FIGS. 4D-1 and 4D-2 are bird's-eye views of each state.

X-rays scattered to the side outside the box are not reduced during surgery compared to above or below. This is due to the fact that there is a penetration port on the side of the box that penetrates human tissue, from which side scattered X-rays leak out of the box. The penetration ports are the sleeve structure 9 of the sleeve port 8 at the end of the additional box and the shielding sheet 22 of the patient port 20.

The sleeve structure 9 is an armhole or a glove attached to the sleeve port 8. One typical example of the sleeve structure 9 is a sleeve manufactured by molding a flexible functional material with shielding. The shape of the sleeve is a cone with both ends open. The opening at the base of the sleeve is where it is attached to the short tube of the sleeve port. The tip of the sleeve opens at the wrist size. When not in operation, the tip of the sleeve is closed by deflating with rubber. This shields the sleeve port 8 when not in operation.

On the other hand, shielding sheets 22 are attached to patient ports 20 provided at both ends in the body axis direction of the box. The shielding sheet 22 is a one-piece sheet made of a flexible functional material that provides shielding. One end of the shielding sheet 22 is connected to the box. In preparation for surgery, after the box is installed through the patient, the shielding sheet 22 is installed so as to close off the remaining space of the patient port 20 as much as possible. This shields the patient port 20 when not in operation.

These penetration ports are provided with flexible shielding structures that block the penetration ports when the box is not in operation. Therefore, when the box is not in operation, sideward scattered X-rays generated in the patient's body do not leak out of the box. However, during surgery, scattered X-rays leak out of the box through the penetration port for the following three reasons.

The first reason is that the human body being penetrated moves. In the sleeve port 8, the hands and arms of a medical worker wearing protective gloves during surgery pass through the sleeve structure 9. On top of that, they are working hard to perform medical procedures. Each time this movement occurs, the tip of the sleeve opens. When the tip of the sleeve is opened, scattered X-rays leak out of the box.

The patient's body passes through the patient port 20 during surgery, and a shielding sheet 22 is placed over it. However, the shielding sheet 22 may shift due to movement of the patient. If the position of the shielding sheet 22 is shifted, scattered X-rays will leak out of the box.

The second reason is that human tissue is transparent to X-rays. Body tissue, such as the hands and arms of the medical personnel or the trunk of the patient's body 60, that pass through the penetration port will block a small percentage of the x-rays. Most of the rest is scattered and some is transmitted. That is, a portion of the incident scattered X-rays leaks out of the box due to transmission and scattering by the body tissue that has penetrated the box.

The third reason is when the shielding ability is insufficient due to the high tube voltage of the X-ray source. The sleeve structure 9 and the shielding sheet 22 are made of a flexible functional material with shielding ability. These materials need to be made of soft material in order to perform delicate medical procedures such as manipulations. Both cannot be made thicker due to the operability required for medical practice. That is, there are obvious limits to the shielding ability of these protective devices. The thickness of these general commercially available products is 0.25 mm Pb or less in terms of lead equivalent. However, with this thickness, the shielding ability is generally insufficient when the tube voltage of the X-ray source is, for example, 88 kV or more.

FIG. 4b and FIG. 4c show the extra space in the patient port 20 that allows leakage of scattered x-rays. The black hatched areas are candidates for extra space.

FIG. 4b shows the sleeve port 8 and FIG. 4c shows the patient port 20. Note that, of the patient port 20, FIG. 4c-1 shows the patient's head side, and FIG. 4c-2 shows the patient's lower limb side.

But FIG. 4b and FIG. 4c show the entire area of the candidate extra space. However, in practice, in the medical field, efforts are made to block the free space from this situation.

However, the patient port 20 cannot completely prevent leakage of X-rays because a surplus space is created during surgery and X-rays are transmitted through the human tissue that passes through the surplus space.

Sleeve ports for inserting hands and arms into the sides of the additional shield box are essential for medical professionals to perform procedures inside the box. Additionally, if there is a patient port at the end in the body axis direction, the patient's head and lower limbs can be taken out of the box. This can reduce medical exposure to patients. Moreover, the patient port can eliminate the psychological anxiety of patients due to closed spaces. Therefore, sleeve ports and patient ports are essential.

Given the existence of these penetration ports, it is necessary to consider measures to reduce the leakage of scattered X-rays to the sides. The patient's body is the source of scattered X-rays. A possible solution is to place a structure of another shielding material in the narrow space between the patient's body and the penetration port. However, the distance between the penetration port of the box and the patient's body is short. It is difficult to place large shielding structures here.

The shielding structure is preferably a plate or film made of metal or resin, which is the simplest structure. Shielding structures that can be placed in this area are patient drapes and patient's garment. FIG. 4e shows a normal hanging cloth 18, and FIG. 4f shows a patient's garment 63.

Both are molded sheets of functional material with shielding ability. Furthermore, a hollowed-out portion 39 is provided in both of them by cutting out the portion of the irradiation field 15. Therefore, the normal hanging cloth 18 and patient's garment 63 cannot strictly shield the X-rays scattered above and below from the irradiation field 15.

FIG. 4a shows the state before measures are taken to reduce X-rays scattered laterally outside the box. FIG. 4d shows the state after the countermeasures have been taken. The arrows around the radiation field 15 in FIG. 4a and FIG. 4d indicate the orientation of the scattered X-rays. As in Example 1, the thickness of the arrow indicates the number of photons, and the length indicates the energy of the scattered X-rays. The arrow above the patient's garment 63 in FIG. 4d-1 represents the state after FIG. 4a is shielded by the patient's garment 63. The arrow on the normal hanging cloth 18 in FIG. 4d-2 represents the state after the arrow on the patient's garment 63 is shielded by the normal hanging cloth 18.

Compare the cloths 63 in FIG. 4a and FIG. 4d-1. In FIG. 4d-1, the width of the downward and lateral arrows is smaller. Similarly, the length becomes smaller. This is because the scattered X-rays generated in the patient's body 60 are blocked by the patient's garment 63.

Compare the top of the patient's garment 63 in FIG. 4d-1 and the top of the normal hanging cloth 18 in FIG. 4d-2. The width of the arrow to the side is smaller on the normal hanging cloth 18 in FIG. 4d-2. Moreover, the length in FIG. 4d-2 is smaller. There is no downward arrow on the normal hanging cloth 18 in FIG. 4d-2. The reason for this is that the hanging cloth does not have the ability to shield downwardly directed backscattered X-rays.

On the other hand, the tops of the normal hanging cloth 18 in FIG. 4a and FIG. 4d-2 are compared. The upward primary X-ray black arrow does not change significantly in both width and length. The reason for this is that both the patient's garment 63 in FIG. 4d-1 and the normal hanging cloth 18 in FIG. 4d-2 have a cutout 39 that cuts out the irradiation field 15.

Next, the shielding ability of areas other than the hollowed-out parts of patient's garments or the hanging cloths will be explained. If the patient's garment or the hanging cloth is the same as sheet-like composite absorbing material as the JIS test material described in Example 10, the transmittance of scattered X-rays can be estimated. In other words, it is assumed that the plate is made of a composite absorbent material with total-t=0.4 to 0.5 mm. The transmittance of scattered X-rays through hanging cloth or patient's garments is approximately 1/50th when the tube voltage is 70 kV compared to when there are no such shield. Similarly, when the tube voltage is 50 kV, it is less than 1/200. These are clear from the JIS test results shown in Example 10.

Therefore, even within the range of the above-mentioned protective instrument, the scattered X-rays irradiated to the sleeve port 8 position in the additional shield box 1 become small within a certain range. Similarly, the scattered X-rays irradiated to the patient port 20 position become small, and even smaller if the re-scattered radiation inside the box is included.

Therefore, the additional use of the normal hanging cloth 18 and/or patient's garment 63 has a great effect in reducing the intensity of the sideward scattered X-rays leaking out of the box.

By combining PD with a new PI (hanging cloth and patient's garment) during surgery, it is possible to reduce the intensity of scattered X-rays that leak to the sides from the box's penetration port during surgery. By using composite absorption materials for these, scattered X-rays can be absorbed after being attenuated. This reduces the radiation exposure of medical personnel and patients, and also reduces the protective burden on medical personnel.

### Example 5

### (Bird's Eye View of the Case Combining a New PI as the Third Protection)

FIG. 5 shows a bird's-eye view of two protective devices (PDs), the first and second protection, combined with a new PI (normal hanging cloth 18 and patient's garment 63) as the third protection. The two protective devices (PDs) in FIG. 5 are a high-performance table 2 and an additional shield box 1. The two protective devices (PDs) in FIG. 5 are a high-performance table 2 and an additional shield box 1. The central part of the additional box 1 in FIG. 5 shows the inside as a perspective view. Scattered X-rays generated in the patient's body 60 are divided and their intensity is reduced. Additional box 1 is in charge of the upper part and the side part when not in operation. High-performance table 2 is in charge of the lower part. Furthermore, by arranging composite absorbing materials 72 at various locations, X-rays can be attenuated, and then absorbed by “line energy absorption”. The normal hanging cloth 18 and the patient's garment 63 are mainly used to cover the sides during surgery. The new PI reduces the intensity of scattered X-rays that leak out of the box from the extra space created in the box accessories (sleeve structure 9 and shielding sheet 22) as the human body moves during surgery.

In addition, in FIG. 4, a Strip-type curtain 54, which will be described later, is attached to the patient port 20 in the lower limb region instead of the shielding sheet 22.

The Strip-type curtain 54 is a flexible curtain with shielding ability and many partitions. This partition is a large number of strip-shaped sheets with a small width in the horizontal direction and a large length in the vertical direction. A strip-shaped sheet is suspended from the top of the penetration port. Since the Strip-type curtain 54 has a large number of flexible partitions, it can be easily opened by pushing with a part of the body. Furthermore, the patient port 20 in the lower limb region is separated from the irradiation field 15 by 40 cm or more, and the dose rate of scattered X-rays at this position is small, so the Strip-type curtain 54 may be used.

The high-performance table 2, which is the first protection, is a medical table that allows X-rays to pass through well and reduces scattering. In the high-performance table 2, the primary X-rays pass through the hollow space and then pass through the mesh of the tabletop plate 7 or the transmission plate unit 32 made of a thin sheet such as CFRP and reach the irradiation field 15 without interaction. The irradiation field 15 is the affected area of the patient's body 60.

The top plate tier 30 includes a tabletop plate 7 that supports the weight of the patient and absorbs scattered X-rays from the patient's body. Furthermore, there is the above-mentioned transmission plate unit 32 and absorption plate 31.

The middle tier 34 can reduce the intensity of backscattered X-rays downward from the periphery of the irradiation field by means of a movable slide table 35 and an aperture plate 36.

The opening/closing plate 42 of the bottom plate tier 40 opens only at the position of the irradiation field 15 by controlling the opening angle. Due to these, the high-performance table 2 allows X-rays to pass through well and reduces scattering.

The additional shield box 1 in FIG. 5, which is the second protection, is a built-in FPD type box 17 installed on the high-performance table 2. The box body 4 is divided into two parts. The additional box 1 does not have an opening that communicates with the external space in any three-dimensional direction and surrounds the irradiation field 15 with functional materials of different combinations of types and thicknesses depending on the dose rate. A line-attenuation material 82 with enhanced shielding ability is arranged on the box top plate 3. A medical worker inserts his hands and arms through the sleeve structure 9 while visually checking the inside of the box through the shielded viewing window 6.

This allows medical personnel to perform medical treatment.

The sleeve structure 9 is attached to a sleeve port 8 on the side of the box with the viewing window 6, and through which medical workers pass their hands and arms. The shielding sheet 22 is located on the side of the box in the body axis direction. Below this the patient port 20 that penetrates the patient's body is attached. Since extra space is created from these box-attached instruments as the hands and arms of the medical personnel and the patient move during surgery, X-rays scattered to the sides leak out of the box.

Here, new protective instrument (PI), normal hanging cloth 18 and patient's garment 63, have been added as the third protection. A normal hanging cloth 18 is placed over the patient's body within the box. That is, it is installed so as to surround the upper and lateral sides of the patient's body. The patient's garment 63 is worn by the patient who is the source of scattered X-rays. Their total mass is less than 20 kg. More preferably, it is 10 kg or less. These have a hollowed-out portion 39 in which the irradiation field of the affected area is cut out to a slightly larger size in order to transmit the primary X-rays. Because of the hollowed-out portion 39, scattered X-rays generated upward and downward from the periphery of the irradiation field cannot be effectively shielded. However, if the shielding of the patient's body 60 is taken into consideration, scattered X-rays directed from the periphery of the irradiation field to the sides within the box can be effectively shielded. Thereby, the normal hanging cloth 18 and the patient's garment 63 can reduce the intensity of scattered X-rays leaking laterally outside the box during surgery.

The combination of the above-mentioned high-performance table 2 (first protection), additional shield box 1 (second protection), and PI (third protection) is a composite protective device, instrument, and tools (PDITS).

Combined PDITS can reduce the intensity of scattered X-rays generated in all directions from the patient's body, including during surgery.

By using composite absorbing materials as functional materials in various parts, scattered X-rays can be attenuated, and then absorbed by “line energy absorption”.

By adding the effect of the high-performance table 2 transmitting X-rays well, the air dose rate in a medical room or the like can be reduced.

In other words, it is possible to reduce occupational exposure and protection burden for medical workers. In addition, medical exposure of patients can be reduced.

### Example 6

### (Case Combining New PI and Table or Box)

In Example 6, a case in which a third protection instrument (PI) is combined with either the first protection (table) or the second protection (box), which are protective devices (PDs), will be described. Additional protective instrument (API), which will be described later, may be added to the protective instrument (PI), which is the third type of protection.

As described above, when the first and second protections are combined, it is possible to reduce the intensity of scattered X-rays emitted to the outside in all directions (three directions: upward, sideways, and downward) during non-operation. In addition, when the first to third protections are combined, the intensity of scattered X-rays leaking outside the box on the side during surgery can be reduced. When the high-performance table 2 is added to these, the performance to transmit primary X-rays well is added.

The “case combining any of them” described in Example 6 is inferior in one or both of the following cases compared to the two cases described above. The first is a) the ability to reduce the intensity of scattered X-rays in all directions. The second is b) the ability to transmit primary X-rays well. First, the cases of the first protection and the third protection will be explained. Next, the case of the second protection and the third protection will be explained.

First, consider the case where there is no box, which is the second protection, but there are a table, which is the first protection, and a normal hanging cloth 18 and patient's garment 63, which are the third protection. The normal hanging cloth 18 with the highest lead equivalent on the market has a corresponding thickness of 0.5 mm Pb. If you stack two of these, it will be 1.0 mm Pb. The patient is also asked to wear patient's garment 63. Assuming that the physical burden on the patient due to mass is 20 kg, which is the maximum allowable limit described below, the thickness of both is approximately 2.0 mm Pb. If the energy of the scattered X-rays generated above and to the sides of the patient's whole body is 50 KeV, the intensity can be reduced to about one-fifteenth of that without shielding. In addition to this, there is also the idea of using a thick hanging cloth 19, which will be described later.

However, around the irradiation field 15 of the hanging cloth and patient's garment, there is a hollowed-out part 39 that allows the primary X-rays to pass through. There is nothing to block the forward scattered X-rays generated in the irradiation field 15 and directed upward. Further, since the forward scattered X-rays have high energy, the X-rays generated around the irradiation field 15 cannot be shielded by the normal hanging cloth 18.

In other words, by installing a combination of the table as the first protection and the hanging cloth and patient's garment 63 as the third protection, the intensity of scattered X-rays generated from the patient's body in all directions except for those directed upward from the periphery of the irradiation field can be reduced within a certain range. However, this simple method is not sufficient to protect medical workers from exposure. In particular, it should be noted that forward scattered X-rays reaching the crystalline lens cannot be reduced much. Therefore, additional box 1 is a necessary condition for sufficient shielding of upwardly scattered X-rays.

Next, consider the case where there is no table as the first protection, but there is a box as the second protection, and a third protection. The new PI for this third type of protection considers the case where a commercially available sheet 21 is added to the normal hanging cloth 18 and patient's garment 63.

Since the sheet 21 is placed under the patient, even if the thickness is increased to correspond to the lead equivalent, there is no physical burden on the patient due to its mass. Therefore, it is possible to set the lead equivalent to 1 to 3 mm Pb, which corresponds to the thick hanging cloth 19 described later. That is, at 3 mm Pb, if the energy of scattered X-rays generated downward from the patient's whole body is 50 KeV, the intensity can be reduced to about 1/60th compared to the case without shielding.

However, around the irradiation field of the sheet 21, there is a hollowed-out portion 39 through which the primary X-rays are transmitted. There is nothing to shield the hollowed-out portion 39. Further, since there is no high-performance table 2, there is no aperture plate 36 below the periphery of the irradiation field 15 that can adjust the position in the body axis direction with high precision. Therefore, backscattered X-rays generated in this region and directed downward cannot be blocked. That is, in addition to the box as the second protection and the hanging cloth and patient's garment 63 as the third protection, a sheet 21 is placed under the patient's body. This makes it possible to reduce a certain percentage of the intensity of scattered X-rays in almost all directions. However, scattered X-rays directed downward from the periphery of the irradiation field of the patient's body are not included. This does not change even during surgery. However, this simple method is not sufficient to protect medical workers from exposure. Therefore, a table is a necessary condition for shielding downward scattered X-rays. In addition, the high-performance table 2 is a necessary and sufficient condition for sufficient shielding.

Consider the ability to transmit primary X-rays well when the second protection and third protection described in the previous section are combined. When the sheet 21 as a PI is used instead of the high-performance table 2 as a PD, scattered X-rays directed downward can be blocked except for the periphery of the irradiation field.

However, the sheet 21 cannot be expected to have the effect of increasing the transmission rate of primary X-rays. Even if the sheet 21 were to have an opening in the area of the irradiation field 15 with the cutout 3, the effect of good transmission would not be obtained. The reason is that a normal table under the bed scatters the primary X-rays and at the same time re-scatters the scattered X-rays. Furthermore, since there is no aperture plate 36, the irradiation field 15 becomes larger than necessary. Therefore, in order to transmit primary X-rays well, it is necessary to have a high-performance table 2.

### Example 7

### (Description of the High-Performance Table of Patent Document 2)

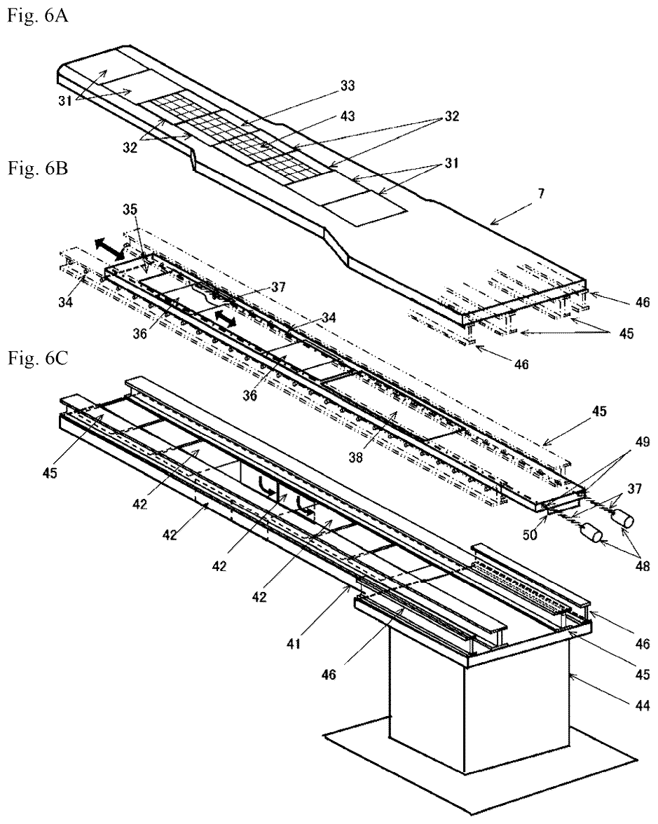

In Example 7, a medical table that allows X-rays to pass through well and eliminates scattering (hereinafter referred to as a “high-performance table”) will be described. High performance tables are one of the tables that is the first protection. Patent Document 2 by the same inventor, describes this high-performance table. FIGS. 6A to 6C show the structure of the high-performance table of Patent Document 2.

The table of a general medical X-ray fluoroscope is a device on which a patient lies, and which supports the patient's weight. The table of the present invention not only supports the body weight, but also has a functional material placed on the upper surface that has the ability to shield the scattered X-rays from the patient placed thereon. Top surface generally refers to the tabletop. The tabletop reduces the intensity of downwardly directed scattered X-rays generated by the patient's body due to the functional material.

Furthermore, the high-performance table 2 of Patent Document 2 is one of the above-mentioned tables. The high-performance table of Patent Document 2 allows primary X-rays to pass through the irradiation field without interacting with substances. In addition, the irradiation field is limited to the minimum necessary aperture size (area) to suppress further generation of scattered X-rays. It also reduces the intensity of scattered X-rays generated downward from the patient's body placed on top. This improves the image quality of the X-ray receiver and reduces the radiation dose rate in a space such as a medical room. Therefore, the exposure dose and radiation protection burden on medical personnel can be reduced.

The high-performance table 2 shown in FIGS. 6A to 6C is composed of the following three stages at maximum. This consists of a top plate tier 30 that supports the weight of the patient and absorbs scattered X-rays, a middle tier 34 that serves as a movable diaphragm for the irradiation field and an absorber, and a bottom plate tier 40 made of low reflection and low scattering material. Certain function can be achieved even with only one or two of these three stages. In FIGS. 6A to 6C, the top plate tier 30 and the bottom plate tier 40 are shown separated into upper and lower parts in order to make the middle tier 34 easier to see. In reality, they are integrated. Its total height is about 10-20 cm. Furthermore, in FIGS. 6A to 6C, the support rails 45 and reinforcing beams 46 are not shown on the top plate tier 30 for easy viewing. Both are shown with a table support 44 on bottom plate tier 40. The middle tier 34 is accommodated by sliding movement into the support rail 45.

The top plate tier 30 of the high-performance table 2 has the following configuration. These are the tabletop plate 7, the absorption plate 31, the transmission plate unit 32, the support rail 45, and the reinforcing beam 46. The tabletop 7 supports the weight of the patient lying down. At the center of the axis of the tabletop plate 7, there is an opening that is long in the body axis direction. The absorption plate 31 is fitted and installed on the support rail 45 below the opening. The absorption plate 31 at the position that will become the irradiation field is removed, and the transmission plate unit 32 is installed. The surface of the mesh 43 of the transmission plate unit 32 is made of a linear material such as CFRP or Al-based material that has high strength and is difficult to absorb X-rays. A thin plate sheet 47 made of CFRP may be used instead of the net 43. After passing through the hollow space, the primary X-rays pass through the net 43 of the top plate tier 30 or the hollow part of the transmission plate unit 32 made of a thin sheet 47 such as CFRP and reach the irradiation field 15 without interaction.

In the high-performance table 2, the irradiation field portion of the bottom plate 41 is cut out to allow primary X-rays to pass through without scattering. In addition, the position of the irradiation field through which the primary X-rays are transmitted, and the aperture size are adjusted using various adjustment functions. These various parts include the transmission plate unit 32 of the top plate tier 30, the slide table 35 and the aperture plate 36 of the middle tier 34, and the opening/closing plate 42 of the bottom plate tier 40.

As a result, in the high-performance table, primary X-rays in the case of the under-tube type are scattered less and transmitted with high positional accuracy due to the aperture function. Due to the ability of the high-performance table 2 to transmit X-rays well, the transmission rate of primary X-rays to the X-ray receiver 10 increases. Therefore, the image quality of the X-ray receiver becomes clearer. If the image quality of an X-ray receiver becomes clearer, an X-ray fluoroscope can be used with lower primary X-ray energy.

In the case of the under-tube type, the upper surfaces of the tabletop plate 7 and the absorption plate 31 of the top plate tier 30 are coated with a functional material. The aperture size of the irradiation field in the body axis direction can be adjusted using a slide table 35 and an aperture plate 36, which will be described later. The aperture size in the body axis direction and in the perpendicular direction can be adjusted using an opening/closing plate 42 and a spacer 33 of the transmission plate unit 32, which will be described later. The load supported by the tabletop plate 7 is supported by the support rail 45 and the reinforcing beam 46, and finally by the table support stand 44.

The middle tier 34 of the high-performance table 2 is composed of a slide table 35, an aperture plate 36, a slide absorption plate 38, and a drive mechanism thereof. The slide table 35 is a flat plate that is long in the axial direction. There is an opening at a part of the center of the axis, and a slide absorption plate 38 is flexibly fixed on the other part. A drive mechanism using a ball screw 37 for the aperture plate 36 is installed inside the slide table 35. A pair (two) of aperture plates 36 can be slid over the opening of the slide table 35 to freely adjust the size of the opening in the axial direction. The slide table 35 installed on the side rollers slides and moves in the axial direction, allowing the position of the opening to be adjusted freely. In this way, the position and size of the aperture in the axial direction of the irradiation field can be adjusted using the slide table 35 and the aperture plate 36.

The bottom plate tier 40 of the high-performance table 2 is composed of a bottom plate 41, an opening/closing plate 42, and a fixing/driving mechanism thereof. The opening/closing plate 42 can be opened/closed by controlling the opening angle using a hinge mechanism or the like. During surgery, the opening/closing plate 42 at the irradiation field position is opened. A mechanical device may be attached to the slide table 35, the aperture plate 36, and the opening/closing plate 42.

By changing the surface material depending on the type or energy of the irradiated X-rays, the generation of scattered X-rays on the bottom plate 41 is reduced, and the tabletop plate 7 attenuates and absorbs the scattered X-rays from human tissues. Further, the aperture size is adjusted by the transparent plate unit 32 of the top plate tier 30, the slide table 35 of the middle tier 34, the aperture plate 36, and the opening/closing plate 42 of the bottom plate tier 40. This aperture should be the minimum size necessary based on the location of the irradiation field and the medical purpose.

In the case of the under-tube type, the high-performance table 2 can reduce the intensity of scattered X-rays generated downward by the patient's body placed on it.

At the top plate tier 30, the tabletop 7 made of a functional material with excellent shielding performance can reduce the intensity of backscattered X-rays directed downward from the irradiation field 15 of the patient's body and its surroundings.

In the middle tier 34, the upper side of the aperture plate 36 is coated with a functional material. Its lower side is covered with a shielding material 81. The slide absorption plate 38 is flexibly attached and fixed to the slide table 35 except for the opening position, and the upper side is covered with a functional material. Thereby, the intensity of backscattered X-rays directed downward can be reduced.

In the bottom plate tier 40, the surface of the opening/closing plate 42 is covered with a shielding material 81 on the outside and a functional material on the inside. Thereby, the intensity of backscattered X-rays directed downward can be reduced.

By placing the composite absorbing material 72 on the functional material described above, scattered X-rays can be attenuated and absorbed by “linear energy absorption.”

The specifications of the wire mesh or thin plate sheet of the transmission plate unit 32 of the top plate tier 30 of the high-performance table 2 will be explained.

First, they must be strong enough to support the weight of the patient's body over the area of the radiation field. The dimensions of the irradiation field are assumed to be 15 cm wide and 15 cm long.

Next, the interaction between materials and X-rays will be described. As shown in Patent Document 1, elements such as hydrogen (H), oxygen (O), carbon (C), Mg, Al, and Si, which have an atomic number of 14 or less, do not absorb X-rays in every energy range.

However, since it is in the Compton region, the interaction is dominated by scattering. Therefore, it is necessary to reduce the area of the wire net or thin plate sheet that is irradiated with X-rays, or to reduce the thickness.

When all of these conditions are satisfied, the wire mesh or thin sheet placed in the irradiation field is preferably made of a material composed of a simple substance or a compound of an element having an atomic number of 14 or less. This is because these elements have difficulty absorbing X-rays.

The strength required for the mesh of the transmission plate unit 32 is to support the weight of the patient's body corresponding to the area of the irradiation field. The tensile strength of CFRP wire is approximately 200 MPa to 400 MPa. With a tensile strength of 200 MPa, a single wire with a diameter of 5 mm can support approximately 400 kg. This strength already exceeds the weight of the human body. However, in order to prevent congestion on the skin surface and the skin from slipping through the mesh and partially falling off and getting caught, it is better to have a large number of wire rods on the mesh surface. In addition, a single wire with a diameter of ϕ2 mm can support approximately 60 kg. Therefore, the wire rod on the net surface of the transmission plate unit has a diameter of 2 mm or more, preferably about 5 mm. The plurality of wire rods is arranged at intervals of 100 mm or less, preferably 50 mm or less. This means that for an irradiation field with an area of 225 cm2, three wires will be installed in each of the width and length directions. These details are shown in Patent Document 2.

Similarly, the strength required for the thin sheet of the transmission plate unit 32 will be considered. The tensile strength of a CFRP thin sheet (thin film) with a thickness of 0.1 mm and a length of 10 cm was investigated. The tensile strength of a thin CFRP sheet with a total cross-sectional area of 0.1 cm2 is 200 to 4,000 kg. Considering the size and area of the irradiation field, a thin sheet with a thickness of 0.1 mm can support the weight of a patient's body. The thinner the thin sheet is, the better, unless there is a strength problem. In general, the thickness of a patient's body is approximately 150 to 300 mm depending on the part, whereas the thickness of the table is approximately 50 mm. In order to improve the image quality of the X-ray receiver, it is desirable that the thickness of the thin plate sheet be less than one-fifth, better still less than one-tenth, of the thickness of the table. To ensure that the thickness is less than one-tenth of the table thickness, the thickness of the thin plate sheet must be less than 0.5 mm. If the thickness of the thin plate sheet is 0.1 mm, this is fully satisfied. These details are shown in Patent Document 2.

### Example 8

### (Description of the Additional Shield Box of Patent Document 3)

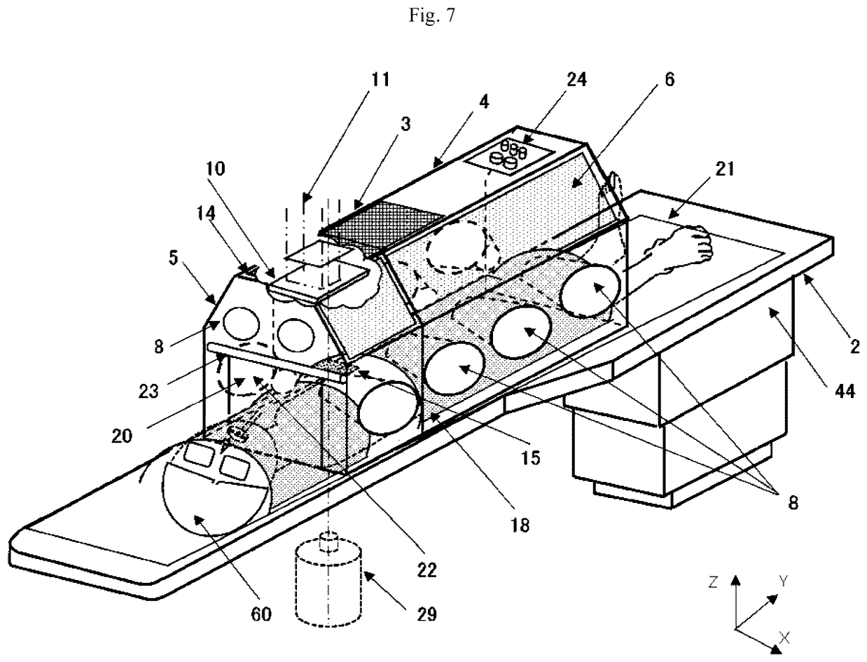

In Example 8, an additional shielding box that reduces exposure and protection load (hereinafter referred to as “additional box”) will be described. The additional box is one of the boxes that is the second protection. Patent Document 3 by the same inventor describes this additional shielding box. FIG. 7 shows the structure of the additional shield box disclosed in Patent Document 3.

General medical X-ray fluoroscopes do not come with an additional shield having a rectangular parallelepiped shape. The box of the present invention surrounds the patient's body above and to the sides, with a functional material placed on the surface or inside of which the scattered X-rays are incident. The box reduces the intensity of upwardly and laterally directed scattered X-rays generated in the patient's body by means of functional materials.

The additional shielding box 1 of Patent Document 3 is one of the boxes and reduces the intensity of various scattered X-rays generated in the patient's body 60 directed upward and laterally. Additional box 1 has no three-dimensional opening that communicates with external space in any direction. The irradiation field is surrounded by functional materials of different types and thickness combinations depending on the dose rate. While viewing the inside of the box through a viewing window with shielding capabilities, medical personnel insert their hands and arms through the sleeves to perform medical procedures. Furthermore, if a composite absorbing material is placed on the inside surface of the box at various locations on the functional material, scattered X-rays can be attenuated, and then absorbed by “line energy absorption”.

The additional box 1 includes a split box type box 16 and a built-in FPD type box 17. FIG. 7 is an overall configuration diagram of the split box type box 16.

Further, FIG. 7 is directed to an X-ray fluoroscope belonging to the X-ray camera type.

On the other hand, in Patent Document 3, a device compatible with a C-arm type X-ray fluoroscope is also devised. The other FPD built-in type box 17 is shown in FIG. 1C of Patent Document 3. In Patent Document 3, a high dose type additional shielding box is devised for cases where the energy of small angle scattered X-rays is higher. This is an option in the box above.

The split box type additional shield box 61 of Patent Document 3 will be explained using the bird's eye view of FIG. 7. The additional box is assembled and installed on the table surrounding the irradiation field such as the trunk of the patient's body. In the split box type box 61, the receiver arm of the X-ray receiver 10 is installed between the split boxes into the receiver port on the box ceiling. Therefore, the box must be divided into two or more parts. The under-tube type X-ray receiver 10 can be assembled on site by sandwiching the receiver arm 11 between the box body 4 and the end box 5, so that it can be stored in the box without an opening. The ceiling of the end box 5 is shielded with a plurality of functional materials to prevent X-rays from leaking.

The additional box 1 in FIG. 7 has a structure that allows the inside to be viewed from the outside space through the viewing window 6. The viewing window 6 is a lead-containing acrylic resin plate or a lead-containing glass plate that is transparent and has shielding ability. The operator performs the surgery using the hands and arms inserted into the box through the sleeve structure 9 while visually checking the inside through the viewing window 6.

The penetration port of the additional box of FIG. 7 and its protective instrument will be explained. The penetration ports of the additional box 1 are the sleeve port 8 and the patient port 20.

A sleeve structure 9 is attached to the sleeve port 8. The sleeve structure 9 is made of a flexible functional material such as a lead-containing arm sleeve. The sleeve port is closed with a flexible sleeve structure that has shielding capabilities. A medical worker inserts hands and arms through the sleeve structure to perform a medical procedure.

The patient port 20 passes through the patient's body 60 in the axial direction of the box. This places the patient's head and limbs parts in a space outside the box. A shielding sheet 22 is attached to the patient port 20. This closes the opening between the box and the human body. The shielding sheet 22 is made of a flexible functional material with shielding ability. Certainly, in Patent Document 3, the shielding sheet 22 is confused with a hanging cloth and is called a “hanging cloth, etc.”. Further, the winding device for the shielding sheet 22 was called a cloth holder together with the cloth. But in the present invention, the contents of “hanging cloth, etc.” are strictly classified and handled separately as follows. A normal hanging cloth 18, a thick hanging cloth 19, and a shielding sheet 22 are distinguished. Further, the hanging cloth holder of Patent Document 3 is referred to as holder 23 in the present invention.

By blocking the penetration port, the box does not have an opening communicating with the external space in any three-dimensional direction during non-operation when no surgery is being performed.

The additional shield box allows the surgeon to perform procedures inside a box with no openings and with shielding ability. By reducing the intensity of scattered X-rays generated above and to the sides of a patient's body, the air dose rate in a medical room or the like is reduced. This will reduce unwarranted occupational exposure of medical workers and avoid unnecessary medical exposure of patients' heads, trunks, limbs, etc. In particular, occupational exposure to the operator's head (eye lens) can be significantly reduced. Furthermore, by using protective clothing and protective glasses that are lightweight, it is possible to reduce the burden of radiation protection on medical personnel. These details are shown in Patent Document 3.

### Example 9

### (Description of Composite Absorbent Material of Patent Document 1)

In Patent Document 1, by the same inventor, a composite absorbent material (CAM) is devised that attenuates scattered X-rays and absorbs them by “linear energy absorption.” The CAM is placed on a surface that is irradiated with scattered X-rays from the patient's body. Here, the CAM efficiently attenuates the incident scattered X-rays and then absorbed by “line energy absorption”. X-rays disappear by converting their energy into kinetic energy such as photoelectrons.

Composite absorption material (CAM) 72 is composed of a low reflection attenuation layer and a multilayer absorption layer. The low reflection attenuation layer is mainly made of lead (Pb) and is arranged in the initial layer of the X-ray incident surface. The multilayer absorption layer behind it is one to three pairs of diffusion absorbers and electron absorbers. A composite absorbing material (CAM) 72 is attached to the surface of a box or table on the X-ray incident side. The total thickness (total-t) of the functional material of the 3 to 4 layers of CAM used in the JIS test is 0.3 to 0.6 mm. Even with such a small thickness, CAM can work if it is made of a multilayer structure consisting of three or more layers with different roles closely stacked on top of each other. Here, CAM attenuates scattered X-rays from scatterers such as human tissues and tables and absorbs them through “linear energy absorption”.

Pb in the first low reflection attenuation layer 80 attenuates most of the incident X-rays and at the same time absorbs a certain percentage of them through “line energy absorption.” The multilayer absorption layer 77 aims to efficiently extinguish X-rays using a pair of a diffusion absorber 78 and an electron absorber 79 by utilizing the specific absorption near the K absorption edge of the material in each energy region.

Here, for the purpose of explaining the contents of this section, μ, μen, and μen/μ are shown in Table 1. Here μ is the linear attenuation coefficient. μen is the linear energy absorption coefficient. This phenomenon of linear energy absorption, expressed as μen, is called “electron absorption.” This phenomenon is a phenomenon in which scattered X-rays are annihilated and converted into electron kinetic energy. Further, μen/μ is the ratio of pen in μ and is called the “electron absorption ratio”. This is a dimensionless value of pen divided by μ, expressed as a percentage. Note that Table 1 excerpts and quotes information from the NIST database of Patent Document 1.

Table 1 quotes the energies of three specific monochromatic and the numerical values of seven elements. Its energies (KeV) are 80, 50, and 30. Moreover, the elements are Pb, W, Ba, Sn, Nb, Mo, and Cu.

Here, the diffusing absorber 78 is an element whose electron absorption rate is less than 70% at a specific monochromatic energy in an arbitrary energy range (for example, 80/50/30 KeV). That is, it is an element with pen/p<70% for a specific monochromatic energy. In Table 1, this is indicated by a frame surrounded by two-dot chain lines.

The electron absorber 79 is an element that absorbs electrons at a high rate, with an electron absorption rate of 70% or more at a specific energy when the diffusion absorber 78 is extracted. In other words, it is an element with specific monochromatic energy and μen/μ>70%. This is indicated in Table 1 by a thick dashed frame. As shown in Table 1, the role of the same element changes depending on its energy value.

The diffusive absorber 78 emits many secondary X-rays (characteristic X-rays, bremsstrahlung X-rays) in various directions due to the unique absorption of the K absorption edge at a specific energy. Along with electron absorption, photons are diffused and pushed back to the adjacent layers. The electron absorber 79 electronically absorbs X-rays in the target energy range, including its secondary X-rays.

The material of the low reflection attenuation layer 80 is mainly Pb. The material of the multilayer absorption layer 77 includes one to three pairs of diffusion absorbers and electron absorbers. For example, at 80 KeV, it is a pair of Sn and Pb. At 50 KeV, it is a pair of Sn and Nb or Mo. At 30 KeV, it is a pair of Nb or Mo and Cu or Fe. The outermost layer is often a flat plate of metal such as Ti alloy or Al alloy, which is the structural material of the box.

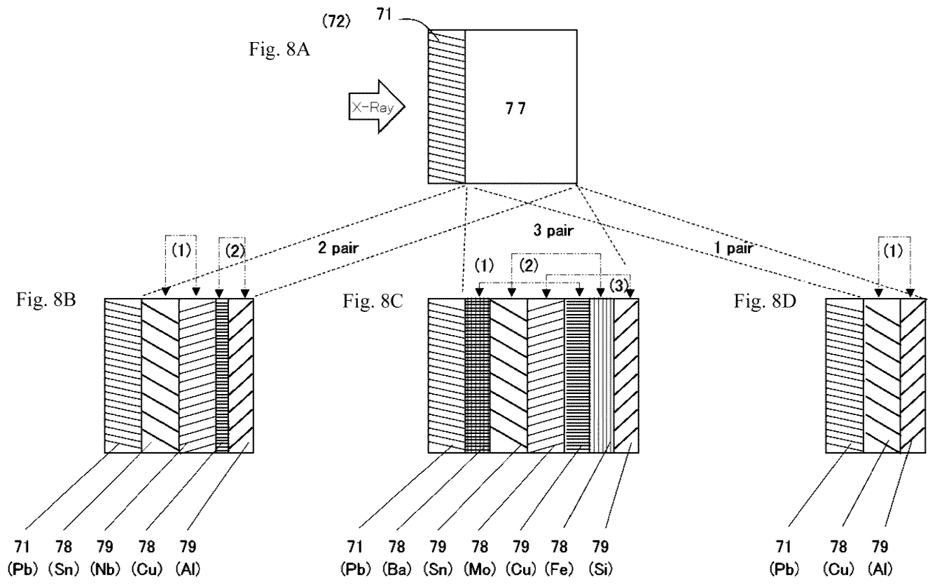

FIGS. 8A to 8D are configuration diagrams of the basic case of the composite absorbent material 72. FIG. 8A shows an overview of the configuration, FIG. 8B shows two pairs and a total of five layers 74, FIG. 8C shows three pairs and a total of seven layers 75, and FIG. 8D shows one pair and a total of three layers 76. Low reflection attenuation layer 80 is Pb in the case of composite absorbing material 72. In the case of the additional composite absorbing material 73, the low reflection attenuation layer 80 is replaced by a line-attenuation material 82 or a low reflection attenuation material 71. Note that the term “pair” refers to the number of pairs of the diffusion absorber 78 and the electron absorber 79 in the multilayer absorption layer 77. In these figures, the pair of diffusion absorber 78 and electron absorber 79 are shown connected by a dashed line, and the numbers (No.) of the pairs in each figure of FIG. 8A to FIG. 8C are indicated by (1) to (3). In the figure, example element symbols are shown in parentheses. An example of a four-layer structure of the composite absorbent material (CAM) 72 is as follows. For example, in the case of flexibility, it is Pb—Sn—Nb—Cu. In the case of rigidity, it is Pb—Sn—Mo—Fe.

### Example 10

### (Explanation of JIS Test Results in Patent Document 1)

Patent Document 1, by the same inventor, reports experimental measurement results of the dose rate of a composite absorbing material (CAM). In Patent Document 1, the transmitted X-ray dose rate of a composite absorbing material having three to five layers in total was measured.

The test method was based on the reverse broad beam conditions (RBB) and narrow beam conditions (NB) of JIS T61331-1 (Protective equipment against diagnostic X-rays). The test specimens were a multilayer test product made by layering thin plates of pure metal (purity>99.9%) for each element. The dimensions of the test piece are 10 cm long×10 cm wide. Here, the type of element, the number of its layers, and the thickness of each layer were used as test parameters. The thickness of the first layer Pb (low reflection attenuation layer) of the multilayer test article is either 0.2 or 0.3 mm. Its total thickness is 0.4-0.6 mm. Table 2 shows the material parameters. In addition, the dose rates of a comparative Pb plate and a blank were measured. The tube voltage of the X-ray source was measured in the range of 50 to 110 kV.

In order to compare the measurement results of different tube voltages, transmittance (%) was calculated from the obtained dose rate. The transmittance is a dimensionless value obtained by dividing the dose rate of the multilayer test product/comparison Pb plate by the dose rate of the blank at each tube voltage and is expressed as a percentage.

For detailed test methods and results, refer to Examples 21 to 23 of Patent Document 1.

Here, the results of seven types of multilayer test piece and two types of comparison Pb plate among the many test results will be introduced. The multilayer test piece is a test piece in which 3 to 4 layers of Pb, Sn, Nb, and Cu are laminated. The thickness of the initial Pb layer is 0.2 mm in each case. The total thickness is 0.35-0.50 mm.

Table 2 shows the extracted seven types of multilayer test piece and two types of comparison Pb plate for comparison. Table 2 shows the types of elements, their respective thicknesses, total thicknesses, average densities, and total masses. Among the multilayer test products in Table 2, No. 2-3 (47 g) on the far left has the largest mass. That is, in Table 2, the mass decreases toward the right. No. 3-7 (36 g) on the far right has the smallest mass.

TABLE 2

| | | Classification | | | | | | | | |
|---|---|---|---|---|---|---|---|---|---|---|
| Item | | Test pieces (T.P.) | | | | | | | Ref. Pb plate | |
| Test pieces. No | | No. 2-3 | No. 3-5 | No. 3-4 | No. 2-5 | No. 2-4 | No. 3-6 | No. 3-7 | 0.3 mm Pb | 0.2 mm Pb |
| Number of layers | | 4 | 4 | 4 | 3 | 3 | 4 | 4 | 1 | 1 |
| Material's | Pb | 0.20 | 0.20 | 0.20 | 0.20 | 0.20 | 0.20 | 0.20 | 0.30 | 0.20 |
| thickness | Sn | 0.10 | 0.05 | 0.10 | — | 0.10 | 0.05 | 0.05 | — | — |
| (mm) | Nb | 0.10 | 0.10 | 0.10 | 0.10 | — | 0.10 | 0.05 | — | — |
| | Cu | 0.10 | 0.10 | 0.05 | 0.10 | 0.10 | 0.05 | 0.05 | — | — |

| | | | | | | | | | |
|---|---|---|---|---|---|---|---|---|---|
| Total thickness | 0.50 | 0.45 | 0.45 | 0.40 | 0.40 | 0.40 | 0.35 | 0.30 | 0.20 |
| (mm) | | | | | | | | | |
| Av. Density | 9.5 | 9.8 | 9.6 | 10.0 | 9.7 | 9.8 | 10.3 | 11.30 | 11.30 |
| (g/cm³) | | | | | | | | | |
| Total mass (g) | 47 | 44 | 43 | 40 | 39 | 39 | 36 | 34 | 23 |

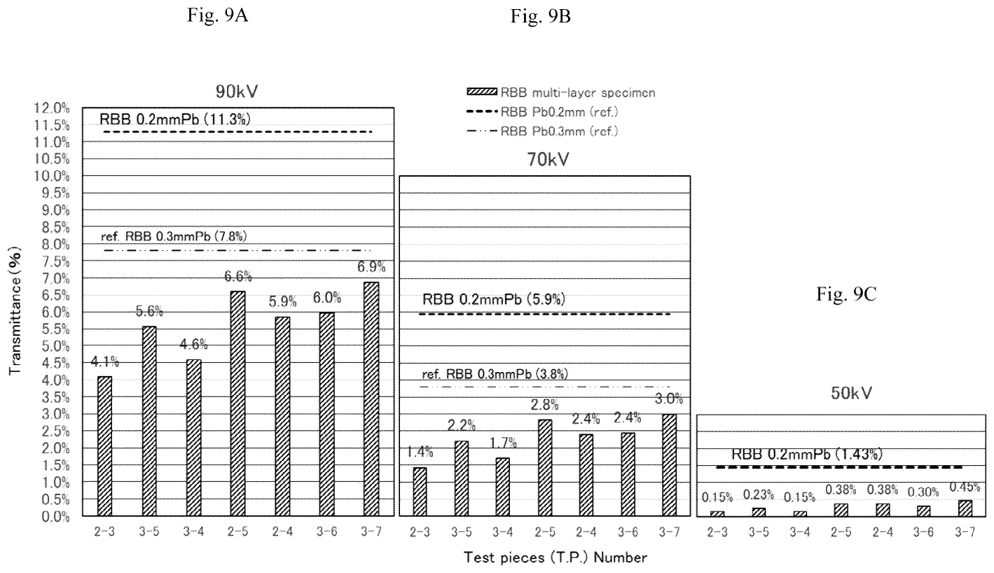

FIGS. 9A to 9C show the transmittance (%) of the multilayer test product obtained in the experiment and the comparative Pb plate. The test piece numbers, and material parameters are shown in Table 2. The tube voltage of the X-ray source is 90 kV in FIG. 9A, 70 kV in FIG. 9B, and 50 kV in FIG. 9C. All measurement schemes result from reverse broad beam conditions (RBB). The transmittance (%) in the figure is shown by a bar graph for the multilayer test article. A comparison Pb plate is shown by the line graph. One of them, 0.2 mm Pb, is shown by a thick broken line. This is because the thickness of the first layer of Pb in all multilayer test products to be compared is 0.2 mm. The other 0.3 mm Pb is indicated by a thin two-dot chain line. This is for reference only. Note that there is no chain double-dashed line for 0.3 mm Pb in FIG. 9C. This is because the dose rate was below the lower limit of quantification of the detector and could not be evaluated at a tube voltage of 50 kV.

The results shown in FIGS. 9A to 9C will be explained. First, the X-ray transmittance of a comparison Pb plate (0.2 mm Pb) and a multilayer test product made of composite absorbing material will be compared. At a tube voltage of 90 kV, the X-ray transmittance was 11.9% for 0.2 mm Pb, while the multilayer test product was 4.1% to 6.9%. At a tube voltage of 70 kV, it was 1.4% to 3.0%, compared to 5.9%. At a tube voltage of 50 kV, it was 0.15% to 0.45%, compared to 1.43%. The transmittance of the multilayer test product made of composite absorbent material was reduced to about half that of the initial Pb layer. Moreover, the mass is not more than twice that of the initial layer Pb. The mass of the functional material per unit area of the multilayer test product used was 3.6 to 4.7 kg/m\hat{ }2.

Next, the correlation between each test piece will be explained with reference to FIGS. 9A to 9C. As mentioned above, the bar graph in FIGS. 9A to 9C is written so that the mass decreases toward the right. In principle, shielding performance is proportional to density. In that case, it is natural for the bar graph in FIGS. 9A to 9C to increase in proportion to the right. However, the bar graph in FIGS. 9A to 9C has irregularities at specific test piece numbers. The same tendency can be seen for this unevenness at any tube voltage.

The following two cases have lower transmittance than the closest left side at tube voltages of 90 kV and 70 kV. They are No.3-4 (total 4 layers, Cu 0.05 mm, others 0.1 mm) and No.2-4 (total 3 layers, no Nb, others 0.1 mm). This is expected to include the effect of Sn.

On the other hand, at a tube voltage of 50 kV, there are the following two. They are No. 3-4 and No. 3-6 (total 4 layers, Nb 0.1 mm, others 0.05 mm). No.2-4 has replaced No.3-6. This is expected to include the effect of Nb.

This tendency for unevenness is considered to be an effect of the multilayer absorbent layers of the composite absorbent material. More specifically, it is thought that the effect of electron absorption by a pair of a diffusion absorber and an electron absorber in a specific energy range was demonstrated here.

The X-ray transmittance of the composite absorbent material was reduced by about half or less by adding a multilayer absorbent layer to the initial Pb layer. This is considered to be an effect of the multilayer absorption layer composed of a pair of a diffusion absorber and an electron absorber. The above was confirmed in the tube voltage range of 50 kV to 90 kV, which corresponds to the effective energy of scattered X-rays generated in the patient's body. That is, the composite absorbing material can effectively reduce the intensity of scattered X-rays generated in the patient's body. These details are shown in Patent Document 1.

### DESCRIPTION OF THE REFERENCE NUMERALS

1. Additional shield box (additional box)
2. High-performance table
3. Box top plate
4. Box body
6. Viewing window
7. Tabletop plate
8. Sleeve port
9. Sleeve structure
10. X-ray receiver
15. Irradiation field
17. Built-in FPD type box
18. Normal hanging cloth
19. Thick hanging cloth
20. Patient port
21. Sheet
22. Shielding sheet
25. Slide
29. X-ray source
30. Top plate tier
31. Absorption board
32. Transmission plate unit
33. Spacer
34. Middle tier
35. Slide table
36. Aperture plate
38. Slide absorption plate
39. Hollowed out part
40. Bottom plate tier
42. Low reflection scattering opening/closing plate
43. Net
44. Table support
45. Support rail
46. Reinforcement beam
47. Thin sheet
51. Hinge mechanism
52. Sealed shield lid
53. Gloveless port
54. Strip-type curtain
55. Closing shield lid
59. Horizontal trolley
60. Patient human body
61. Patient port lid
63. patient's garment
64. Head cover
67. hand take-out port
68. Stopper
69. Shoulder strap
70. Cover
71. Low reflection attenuation material
72. Composite absorbent material
73. Additional composite absorbent material
77. Multilayer absorbent layer
78. Diffusion absorber
79. Electron absorber
80. Low reflection attenuation layer
81. Shielding material
82. Line-attenuation material

1. A compound protective equipment and instrument, wherein
in a radiation protective device of X-ray fluoroscopy equipment of a medical under-tube fluoroscopy,
a planar table serving as a first protection and having an upper surface is installed, a functional material with a shielding capability against a scattered X-ray from a patient placed on the planar table being disposed on the upper surface, and a box serving as a second protection and having a rectangular parallelepiped shape is installed on the table, the functional material with the shielding capability being disposed either on a surface or in inside of the box, the scattered X-ray being incident on the surface,
an X-ray receiver without an opening incapable of reducing transmission of an X-ray is accommodated in the box,
all directions of a body of the patient are surrounded with a structure, either on a surface or in inside of which the functional material with the shielding capability is disposed, and thus
intensity of scattered X-rays generated from the body of the patient including a periphery of an irradiation field and emitted to 360 degrees including all directions of up, down, left, and right directions is reduced.

2. The compound protective equipment and instrument according to claim 1, wherein
a planar table, a box, and at least one of three protective instruments are installed,
the planar table serving as a first protection and having an upper surface, on which a functional material with a shielding capability against a scattered X-ray is disposed,
the box serving as a second protection and having a rectangular parallelepiped shape, the functional material with the shielding capability being disposed either on a surface or in inside of the box, the scattered X-ray being incident on the surface,
at least one of the three protective instruments serving as a third protection and including a hanging cloth to be worn covering over a body of the patient, a garment to be worn on a trunk of the patient, and a head cover to be worn on a head of the patient, so as to be worn on a patient to protect a medical worker from exposure, the three protective instruments each including the functional material that is flexible either on an incident-side surface of the scattered X-ray or in inside,
an X-ray receiver without an opening incapable of reducing transmission of an X-ray is accommodated in the box,
all directions of the body of the patient are surrounded with a structure, either on a surface or in inside of which the functional material with the shielding capability is disposed, and thus
intensity of scattered X-rays generated from the body of the patient and emitted to all directions during surgery is reduced.

3. A compound protective equipment and instrument, wherein
in a radiation protective device of X-ray fluoroscopy equipment of a medical under-tube fluoroscopy,
a planar table and at least one of three protective instruments are used in combination,
the planar table serving as a first protection and having an upper surface, on which a functional material with a shielding capability against a scattered X-ray is disposed,
the three protective instruments including a hanging cloth, a garment, and a head cover, each of which includes the functional material either on a surface or in inside, the scattered X-ray being incident on the surface,
all directions of the body of the patient are surrounded with a structure, either on a surface or in inside of which the functional material with the shielding capability is disposed, and thus
intensity of scattered X-rays generated to all directions is reduced, excluding scattered X-rays directed upward from a periphery of an irradiation field from scattered X-rays generated from the body of the patient and emitted to all directions.

4. A compound protective equipment and instrument, wherein
in a radiation protective device of X-ray fluoroscopy equipment of a medical under-tube fluoroscopy,
a box, at least one of three protective instruments, and a sheet are installed,
the box serving as a second protection in which a functional material with a shielding capability against a scattered X-ray is disposed,
the at least one of three protective instruments, either on a surface or in inside of which the functional material is disposed, the three protective instruments including a hanging cloth, a garment, and a head cover, the scattered X-ray being incident on the surface,
the sheet being positioned under a body of a patient, positioned between the box and a planar table on which the body of the patient is placed, and positioned between the body of the patient and the table,
an X-ray receiver without an opening incapable of reducing transmission of an X-ray is accommodated in the box,
all directions of the body of the patient are surrounded with a structure, either on a surface or in inside of which the functional material with the shielding capability is disposed, and thus
intensity of scattered X-rays generated to all directions is reduced, excluding scattered X-rays directed downward from a periphery of an irradiation field from scattered X-rays generated from the body of the patient and emitted to all directions during surgery.

5. The compound protective equipment and instrument according to claim 1, wherein
the table serves as a high-performance table in which a net or a thin plate sheet containing either a single element having an atomic number equal to or smaller than 14 or a compound of elements on a top plate tier is installed in an area of the irradiation field to be irradiated with a primary X-ray, and the primary X-ray is allowed to transmit through either the net or the thin plate sheet for a top plate to be capable of reducing absorption and scattering of the X ray.

6. The compound protective equipment and instrument according to claim 5, wherein
the table includes a bottom plate or a middle tier below the top plate tier,
in the area of the irradiation field to be irradiated with the primary X-ray, the bottom plate or the middle tier is cut out for the X ray to pass through a hollow free space, and thus
the table reduces the absorption and scattering of the X ray.

7. The compound protective equipment and instrument according to claim 5, wherein
in the table, as a material of a surface of the top plate tier to be irradiated with the scattered X-ray from the body of the patient placed on the table, at least one functional material with the shielding capability against the scattered X-ray to be irradiated on each area is disposed, and thus the scattered X-ray is attenuated and absorbed to reduce re-scattering of the X ray.

8. The compound protective equipment and instrument according to claim 1, wherein
the box having the rectangular parallelepiped shape serves as an additional shield box, to which a device with the shielding capability and for visually checking inside either in close proximity or remotely and a device with the shielding capability and for operating the inside either in close proximity or remotely are attached.

9. The compound protective equipment and instrument according to claim 8, wherein
the box having the rectangular parallelepiped shape includes a member in which either a structural material of the box including the functional material or the functional material is disposed on a surface layer of the box, and serves as an additional shield box in which the member is installed to three-dimensionally surround the irradiation field of the patient.

10. The compound protective equipment and instrument according to claim 8, wherein
the device, of the box, for visually checking the inside in close proximity includes a transparent box including either a viewing window or a structure of a transparent lead-containing acrylic resin, the viewing window including, form either a ceiling surface of a wall surface of the box, at least one glass plate with a shield of see-through transparency or a resin plate with a shield, the structure of the transparent lead-containing acrylic resin including a shield with the shielding capability and having either the rectangular parallelepiped shape or a semi-cylindrical shape of either an integrated type or a split type divided into at least two parts.

11. The compound protective equipment and instrument according to claim 8, wherein
the device, of the box, for operating the inside in close proximity includes either at least one flexible sleeve structure or a strip-type curtain, the at least one flexible sleeve structure being attached to a sleeve port on a wall surface of the box, the strip-type curtain including a large number of flexible partitions in a strip-shaped flexible shielding sheet installed to be hung from a top of the sleeve port having a rectangular shape provided on either a partial width or an entire width of a side surface on which the viewing window of the box is provided.

12. The compound protective equipment and instrument according to claim 8, wherein
into the box having the rectangular parallelepiped shape, the body of the patient is caused to penetrate in a body axis direction through a patient port on an end face of the box to place areas except for the periphery of the irradiation field of the patient in an external space, a flexible shielding sheet with the shielding capability is hung from a top of the patient port, or a strip-type curtain is installed to be hung from the top, the strip-type curtain including a large number of flexible partitions in a strip-shaped flexible shielding sheet including a flexible functional material with the shielding capability and at least two partitions that are cut in a vertical direction toward a bottom end, and thus an opening incapable of reducing the transmission of the X-ray is blocked.

13. The compound protective equipment and instrument according to claim 1, wherein
the functional material serves as a composite absorption material in which at least three layers are closely stacked into a multilayer, the at least three layers including a first layer, on which the X ray is incident, and which serves as a low reflection attenuation layer of an element having an atomic number equal to or larger than 82, and a second layer or lower including a multilayer absorption layer including a pair of a diffusion absorber and an electron absorber.
